(19) United States
(12) Patent Application Publication
(10) Pub. No.: US 2002/0146753 A1
(43) Pub. Date: Oct. 10, 2002

(54) Autoantibodies to glucose-6-phosphate isomerase and their participation in autoimmune disease

(76) Inventors: Henrik Ditzel, San Diego, CA (US); Dennis R. Burton, La Jolla, CA (US); Monica Schaller, San Diego, CA (US)

Correspondence Address: Schwegman, Lundberg, Woessner & Kluth, P.A., P.O. Box 2938, Minneapolis, MN, 55402, US

(21) Appl. No.: 09/828,708

(22) Filed: Apr. 6, 2001

(51) Int. Cl. G01N 33/53; G01N 33/537; G01N 33/543; C07K 16/40

(52) U.S. Cl. 435/7.92; 530/388.26

(57) ABSTRACT

It has been discovered that one of the causes of human rheumatoid arthritis is an autoimmune reaction to human glucose-6-phosphate isomerase. While human glucose-6-phosphate isomerase is a normal constituent of living tissue, and the underlying reasons for the autoimmune reaction are not understood, this discovery enables effective treatment of the disease, especially when the human immune reaction is the primary cause of the rheumatoid arthritis. The human antibody, anti-glucose-6-phosphate isomerase IgG, can be used to develop immunopolypeptides having binding capacity with the antigen. Antibodies and antibody fragments to the antiglucose-6-phosphate isomerase IgG, antisense oligonucleotides, conjugates of human GPI with cytotoxic agents, immobilized human GPI may be used to ameliorate or eliminate the immune reaction. The peptide, nucleotide products as well as methods of diagnosis and treatment are provided.

### FIELD OF THE INVENTION

The invention relates to the discovery that human glucose-6-phosphate isomerase (GPI) is an antigen involved in the development of human autoimmune diseases such as rheumatoid arthritis. The human antibody to GPI, fragments of that antibody, antisense oligonucleotides, as well as GPI itself can be used for diagnosis and treatment of human autoimmune diseases.

### BACKGROUND OF THE INVENTION

Autoimmune disease such as rheumatoid arthritis (RA) is a chronic inflammatory joint disease that eventually leads to the destruction of the joint architecture. See Feldmann, M., Brennan, F. M. & Mainin, R. N. Rheumatoid arthritis. Cell 85, 307-310 (1996); and Hale, L. P. & Haynes, B. F. Pathology of rheumatoid arthritis and associated disorders. In William J. Koopman (ed.) Arthritis and allied conditions: a textbook of rheumatology. Williams & Wilkins, Philadelphia (1997). The synovial lining layer of the inflamed joints is thickened due to increased proliferation of synoviocytes and the synovial stroma is invaded by predominantly mature memory CD4+T lymphocytes, but also significant number of B cells, cytotoxic T cells, monocytes and dendritic cells. In part because of the association of rheumatoid arthritis with certain HLA-DR shared epitope-bearing alleles, CD4+T cells have long been considered to play a central role in the origin and propagation of the joint inflammation of RA.

However the exact mechanism remains controversial. It has been hypothesized that arthritogenic antigens presented by RA-associated HLA-DR molecules activate selfreactive antigen-specific T cells, thus initiating synovial inflammation. A candidate arthritogenic antigen in rheumatoid arthritis has not however been identified. Although many presume that the antigen should be specifically expressed in the joint, an alternative is that an ubiquitously expressed antigen is modified and exposed in the joints as a neoepitope. See Lehmann, P. V., Forsthuber, T., Miller, A. & Sercarz, E. E. Spreading of T-cell autoimmunity to cryptic determinants of an autoantigen. Nature 358, 155-157 (1992). Antibodies have been considered as epiphenomena in RA, but recent results from animal models of this disease indicate that they may play a more active role than previously considered. See Holmdahl, R. et al. Type II collagen autoimmunity in animals and provocations leading to arthritis. Immunol. Rev. 118, 193-232 (1990); Korganow, A.-S. et al. From systemic T cell self-reactivity to organ-specific autoimmune disease via immunoglobulins. Immunity 10, 451-461 (1999). Several animal models for RA have been developed including a recent mouse model. See for example PCT publication WO 00/64469 or Matsumoto, I., Staub, A., Benoist, C. & Mathis, D., Arthritis provoked by linked T and B cell recognition of a glycolytic enzyme. Science 286, 1732-1735 (1999). However, there is no indication that these models are predictive of RA in humans. Nor is there any indication in humans regarding whether postulated antigens are specifically expressed in joints or are ubiquitously expressed. Moreover, researchers have recently criticized the animal models as not indicating the difficulties, etiologies, causes and symptoms of RA in humans. See R. Holmdahl, Arthritis Res., 2000;2(3)175-8.

Therefore, there is a need to demonstrate directly in humans the role of certain antigens and antibodies in the onset and course of autoimmune disease, such as rheumatoid arthritis. If antigens do play a part, there is also a need to determine whether such antigens are specifically produced by afflicted tissues or are ubiquitously produced. There is a further need to determine in humans whether such antigens are responsible for the immune response associated with rheumatoid arthritis or are a result of it. There is also a need to develop methods in human for diagnosis and treatment of rheumatoid arthritis which are based upon these discoveries.

### SUMMARY OF THE INVENTION

These and other needs are achieved by present invention, which is directed to immune agents involved in human autoimmune disease such as rheumatoid arthritis, therapeutic agents interacting with these immune agents, and the diagnosis and treatment of autoimmune diseases such as rheumatoid arthritis. The immune agents according to the invention include the antigen, human glucose-6-phosphate isomerase (GPI) and a human autoimmune antibody to GPI, anti-GPI antibody (AGPI Ab). The invention is also directed to therapeutic agents such as an immunopolypeptide relating to AGPI Ab, which includes any antibody fragment of AGPI Ab. The therapeutic agents also include an anti-idiotypic antibody to AGPI Ab, a GPI-cytotoxic agent conjugate, a GPI epitope conjugated to a cytotoxic agent, an antisense oligonucleotide relating to AGPI Ab production, immobilized GPI, and pharmaceutical compositions of these therapeutic agents. The present invention is further directed to the use of these therapeutic agents in diagnosis and treatment of human autoimmune disease such as rheumatoid arthritis. Unless otherwise indicated, all references to autoimmune disease, rheumatoid arthritis, antibodies of any kind, antibody fragments, proteins, enzymes, GPI, polypeptides and the like include the understanding that such references are directed to, or are based upon, a human origin irrespective of whether the term “human” or its acronym “h” is stated.

The anti-GPI antibody (AGPI Ab) of the invention is a human antibody that is immunologically specific for human GPI. This antibody may be a member of any human antibody class such as IgG, IgM, IgE and IgA. The preferred human antibody class is IgG. AGPI Ab has significant homology to an amino acid sequence of a human antibody of any of the foregoing classes, and includes any of the CDR and framework region sequences as given by the name matches of SEQ ID NO's: 1-14 (l and h chains together). Preferably, AGPI Ab is a purified or isolated monoclonal antibody having a human IgG structure with its light and heavy CDR and framework regions as given by any of SEQ ID NO's: 1-14. Additional preferred embodiments of the AGPI Ab are the full human antibody with a modified Fc region. The modifications include complement binding site modification or destruction and Fc binding site modification or destruction. These modifications can be produced by site directed mutagenesis.

The immunopolypeptide of the invention is a human based amino acid sequence having a segment that has significant homology (defined herein as at least about 80% homology, preferably at least 90% homology, especially preferably at least 95% homology, most preferably at least 99% homology) with at least any of the 42 CDR amino acid sequences given in FIG. 4A. Those 42 CDR sequences have SEQ ID NO's: 15-56. Preferably, the immunopolypeptide incorporates a segment that has at least 80% homology with any of the segments having SEQ ID NO's: 1-14 of FIG. 3AL and FIG. 3AH. The immunopolypeptide may have a size equal to such a CDR segment or may incorporate such a CDR segment in a larger polypeptide. Preferably, the CDR's for the immunopolypeptide are selected as triplets so that the immunopolypeptide will contain at least three CDR sequences. Preferably, the immunopolypeptide amino acid sequence also includes any of the framework regions having the amino acid sequences given in FIG. 4B. Preferably, the CDR and framework sequences are matched from one Fab or Fv fragment as shown in FIG. 3AL and FIG. 3AH. Variants of the amino acid units of these CDR and framework regions are also included. In this preferred form, the immunopolypeptide incorporates an amino acid sequence that is substantially identical to any of SEQ ID NO's 1-14 (separate 1 and h Fv chains). Also included are the fragments of AGPI Ab. These fragments include the Fab, Fab', Fv, Fl, Fh, Fd fragments as well as fusion proteins with these fragments and a carrier protein. Also included are the single chain antibodies, diabodies, linear antibodies, and multispecific antibodies based upon AGPI Ab.

The AGPI Ab and immunopolypeptide of the invention demonstrate significant immunobinding, preferably high affinity binding, with human glucose-6-phosphate isomerase (GPI). In particular, the human AGPI Ab and human based immunopolypeptide of the invention immunoreact with one or more epitopes of human GPI.

The anti-idiotypic antibody, preferably a monoclonal antibody to AGPI Ab and the second immunopolypeptides of the invention exhibit immunospecific binding with AGPI Ab. The anti-idiotypic antibody is obtained by inserting the CDR's of an antiidiotypic antibody to AGPI Ab into a human IgG consensus framework. The antiidiotypic antibody can be obtained from a human host who has naturally developed such antibodies or may be obtained from a non-human mammalian host through an immunization process. The anti-idiotypic antibody reacts with the variable region of AGPI Ab. The second immunopolypeptides have the same design and variation as the immunopolypeptides described above. The second immunopolypeptides have CDR's that follow the CDR sequences of the anti-idiotypic antibody to AGPI Ab. The second immunopolypeptides react with the variable region of AGPI Ab. Preferably, the antiidiotypic antibody and second immunopolypeptide bind to one or more CDR's or sequences closely associated with the CDR's, of human AGPI Ab.

The anti-idiotypic antibody may be obtained by a phage process as described below, a hybridoma process using an animal immunized with AGPI Ab, a transgenic animal carrying a human immune system, or a humanized animal carrying human B cells. Preferably, the animal is a laboratory mammal, preferably a mouse, rat or rabbit.

The antisense oligonucleotide of the invention is designed to hybridize with a native human polynucleotide sequence encoding the AGPI Ab or a native human polynucleotide sequence encoding GPI. Preferably it is the complement of the MRNA encoding the AGPI Ab or GPI. Especially preferably, the antisense oligonucleotide is designed to have a significantly long half-life in the blood stream of a patient. Especially preferably, the antisense oligonucleotide is formulated in a sustained release dosage form. Preferably, an antisense oligonucleotide to a polynucleotide encoding GPI is administered by a titration procedure in which the dosage of the antisense oligonucleotide is increased until the concentration of GPI in sera is lowered to a normal level.

The conjugate of the invention is GPI or an epitope of GPI bound, complexed or conjugated to a cytotoxic agent. Such agents include radioactive organic molecules, and known cytotoxic agents.

The immobilized GPI is human GPI covalently bound to a support material. It may be used to extracorporeally remove AGPI Ab from a patient's blood stream.

The invention also provides nucleotide sequences encoding the individual V_{H} and V_{L} chains of the AGPI Ab of FIG. 3. These nucleotide sequences are given in FIG. 5A and 5B. Nucleotide sequences encoding the remaining immunopolypeptides of the invention can be constructed from segments of these V_{H} and V_{L} nucleotide chains in combination with known mammalian consensus constant and framework regions. These consensus nucleotide sequences are known in the art.

Vectors encoding any of the individual AGPI Ab or immunopolypeptide sequences described above are also included in the invention. These include plasmids, phages, viruses and nucleotide segments for insertion into prokaryotic and eukaryotic cells. In particular, a vector for insertion of DNA encoding the immunopolypeptide into Chinese hamster ovary (CHO) cells is preferred. The vectors may appropriate regulatory sequences for expression, including but not limited to promoter, operator and transcription element intron sequences.

The recombinant cells of the invention include bacterial host cells, which have been transformed with a phage embodiment of the phage library. Also included are eukaryotic host cells and mammalian host cells such as CHO cells, which have been transfected with an expression vector carrying a DNA sequence for the immunopolypeptide of the invention.

The process of the invention includes any recombinant technique to express the AGPI Ab, immunopolypeptide or humanized chimeric antibody, or fully human antibody of the invention. The corresponding DNA sequence may be inserted into an expression vector, that vector used to transfect an appropriate host cell and the host cell cultured to provide the desired AGPI Ab, immunopolypeptide or humanized chimeric antibody, or fully human antibody.

The diagnostic method of the invention is based upon the highly specific immune reaction to human GPI demonstrated by patients with rheumatoid arthritis. The method involves treating a patient's blood serum with an effective amount of GPI and analyzing for the GPI-AGPI Ab complex using any of the standard antibody complex detection methods.

The therapeutic methods of the invention include treatment of a patient through use of the following protocols:

a) use of humanized chimeric antibodies, or fully human antibodies to AGPI Ab;

b) use of an immunopolypeptide to block the AGPI Ab—GPI binding;

c) use of an antisense oligonucleotide;

d) use of GPI or a GPI epitope bound to a cytotoxic agent;

e) use of immobilized GPI to extracorporeally remove AGPI Ab from the patient's blood stream; and

f) use of GPI to induce tolerance by desensitization.

When these methods involve invasive techniques, administration of an effective amount of the treatment agent, preferably as a pharmaceutical composition, will provide the desired effect.

### BRIEF DESCRIPTION OF THE DRAWINGS

FIG. 1 shows the serum antibody reactivity of patients tested as determined by ELISA (A) and Western Blot (B).

FIG.1 (A), (A) serum antibody reactivity against GPI as measured by ELISA for 69 patients with RA, 17 patients with Lyme's arthritis, 22 patients with Sjögren's syndrome and 107 normal healthy donors. Sera, diluted 1:50 in PBS, were allowed to react for 2 hr with purified GPI (5 μg/ml) coated on ELISA plates. Bound antibody was detected with an alkaline phosphatase-conjugated F(ab')₂ goat anti-human IgG antibody, and visualized with nitrophenol substrate by reading absorbance at 405 nm. Patient samples with OD 405 values of more than two standard deviations above the mean of the control normal donor values (>1.33) were considered positive. (B) The sera were also tested for reactivity against purified GPI by Western blotting. 1B depicts staining of a 55 kDa GPI band by 5 RA sera (lanes 4-8), whereas no staining was observed by normal sera (lanes 23) or by the secondary anti-human IgG antibody alone (lane 1).

FIG. 2 shows a bar graph of the specific immunoreactivity of the AGPI Ab of the invention.

In FIG. 2 the graph summarizes human monoclonal IgG antibody Fab fragments bind specifically to GPI. To evaluate specificity, the Fabs were tested for binding to GPI, BSA, HIV-1 gp120 and the Fc fragment of IgG, by ELISA. The anti-gp120 Fabs L17 (5 μg/ml) and rabbit anti-GPI antibody (1:200) were used as negative and positive controls, respectively.

FIGS. 3AL, 3AH, 3B and 3C show the amino acid sequences of the variable domains (CDR and Framework regions) of the light and heavy chains of the AGPI Ab of the invention. In FIGS. 3AL and 3AH the various frame work and CDR regions of the sequences, ID SEQ NO's: 1-14, are indicated by separations; however, the sequences are contiguous. For example, the S residue of the A4 heavy chain FRI is bound directly to the S residue of the CDR1 region.

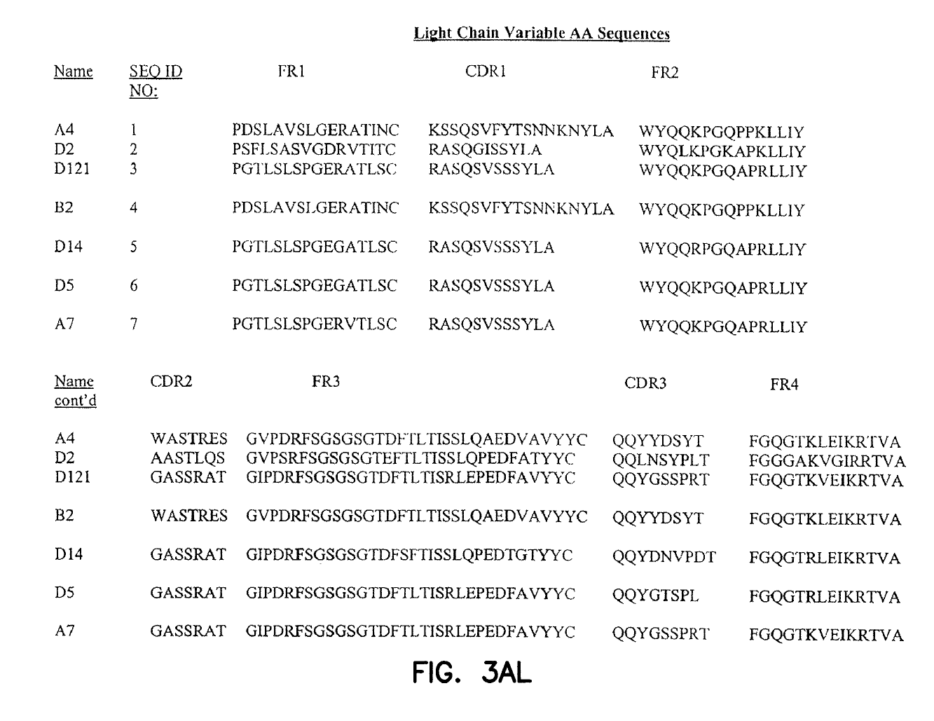

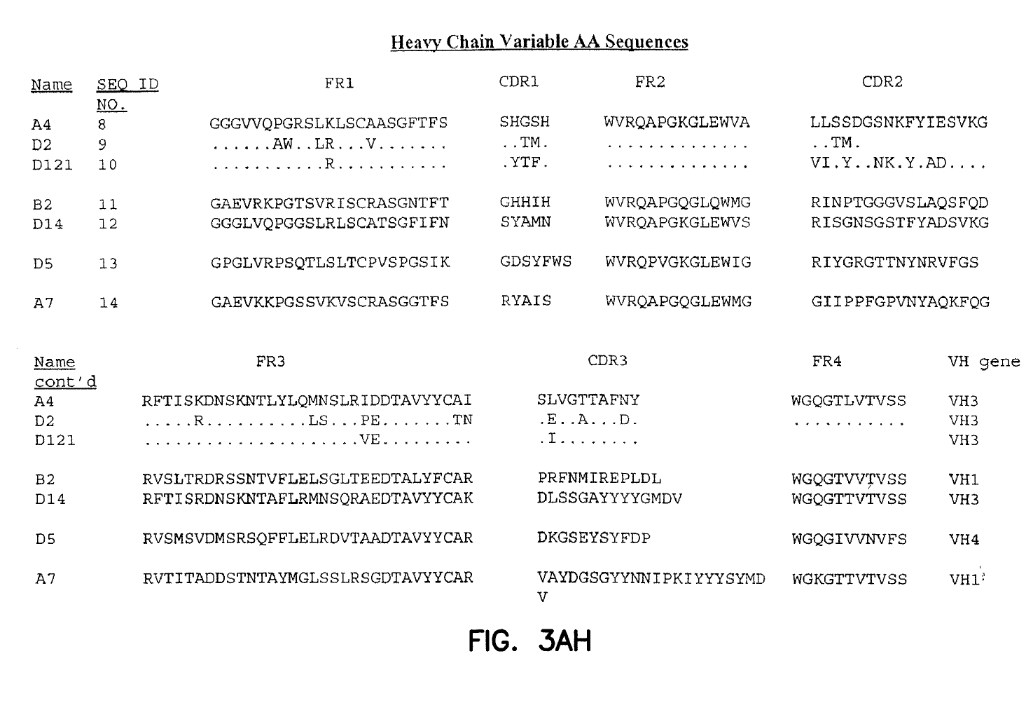

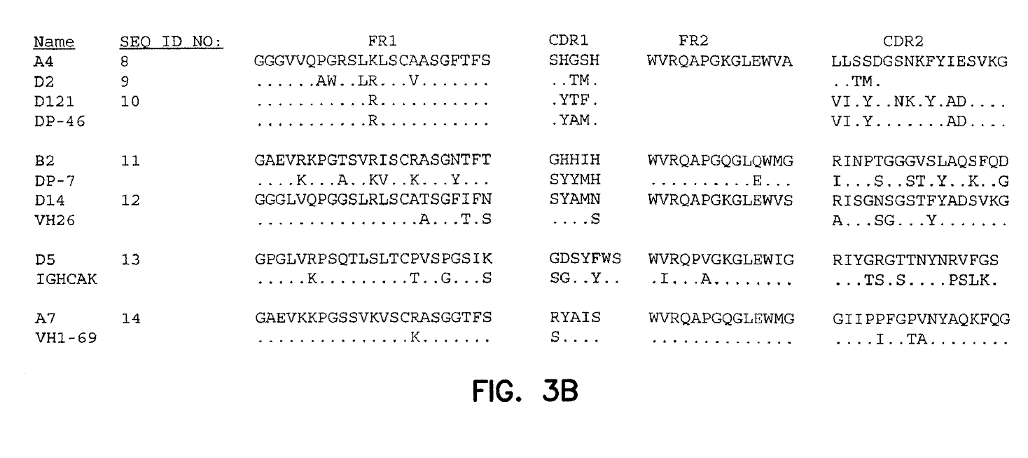

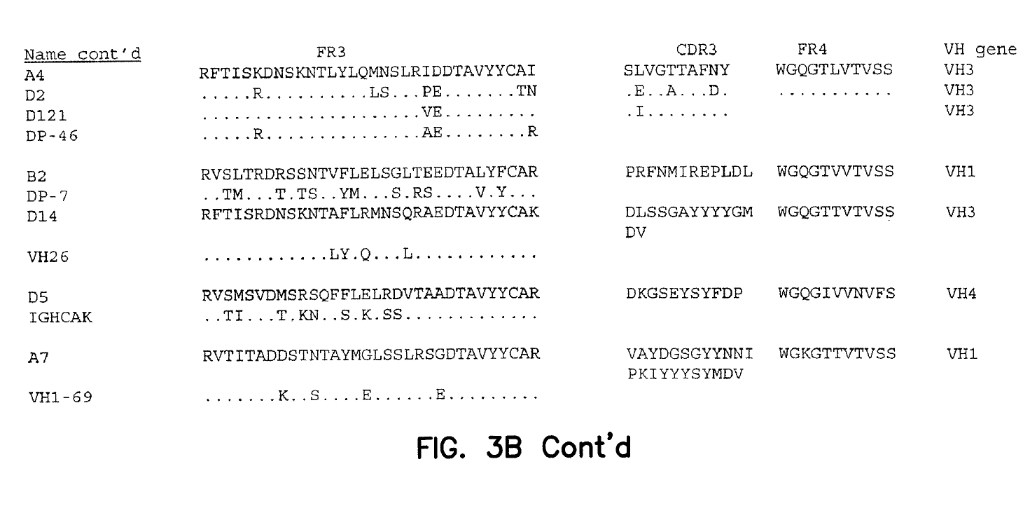

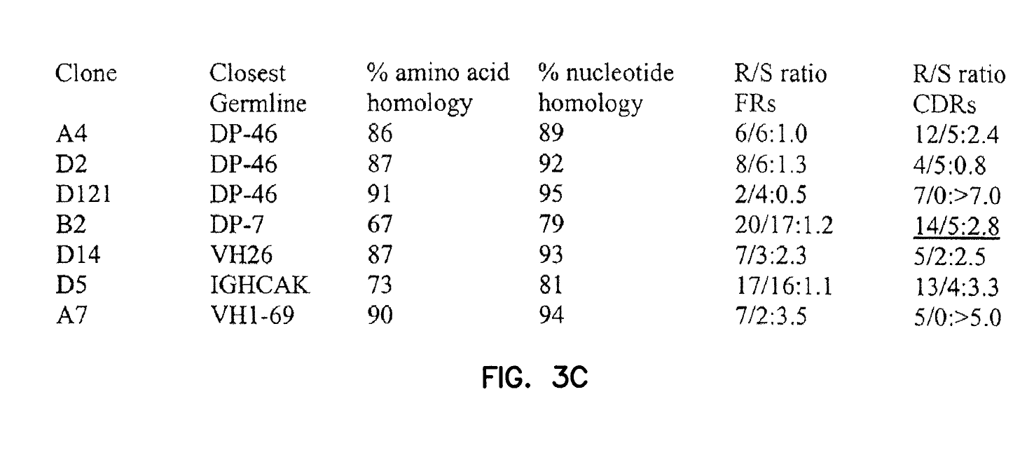

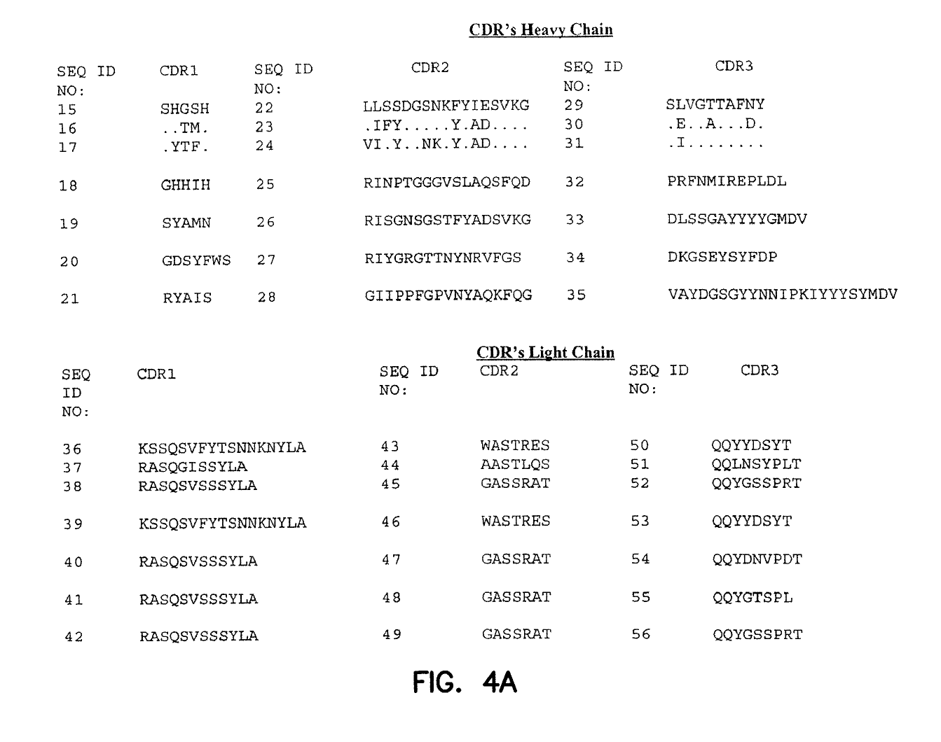

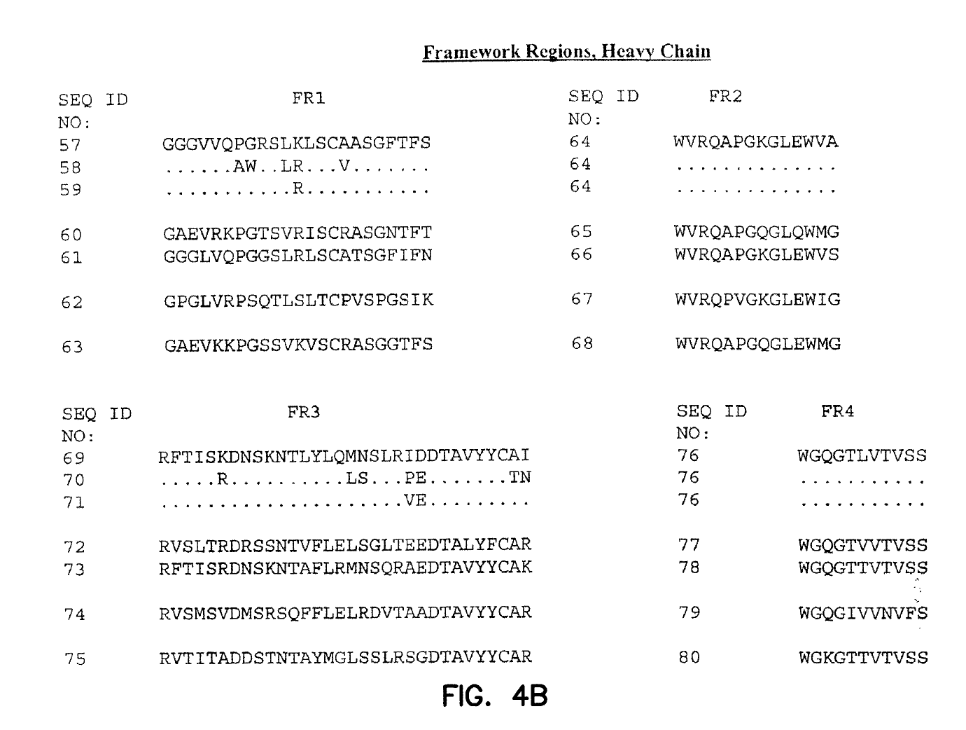

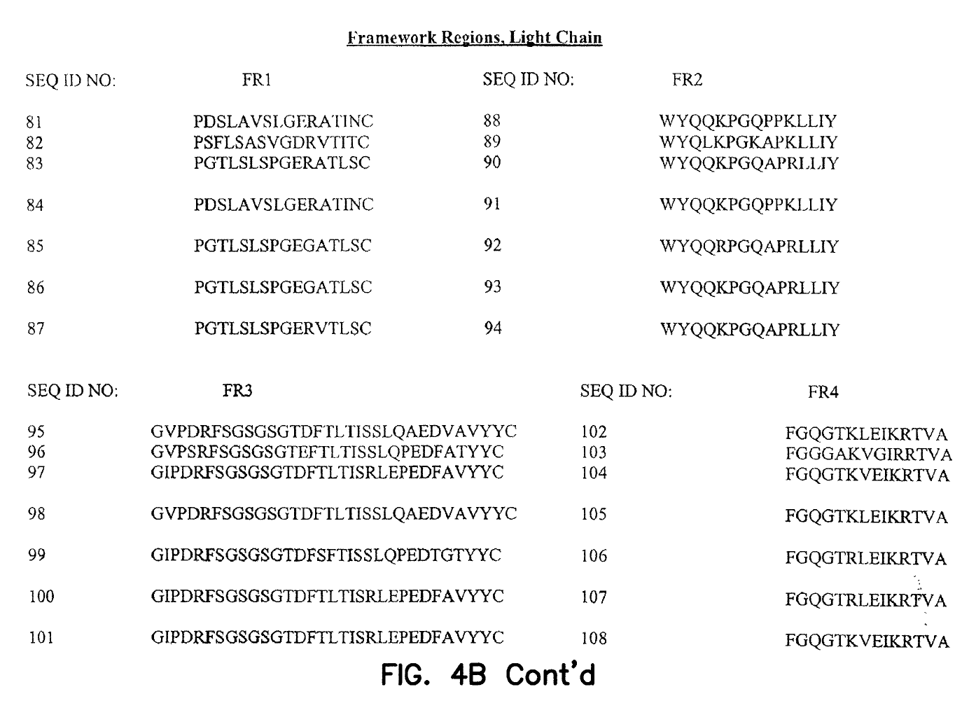

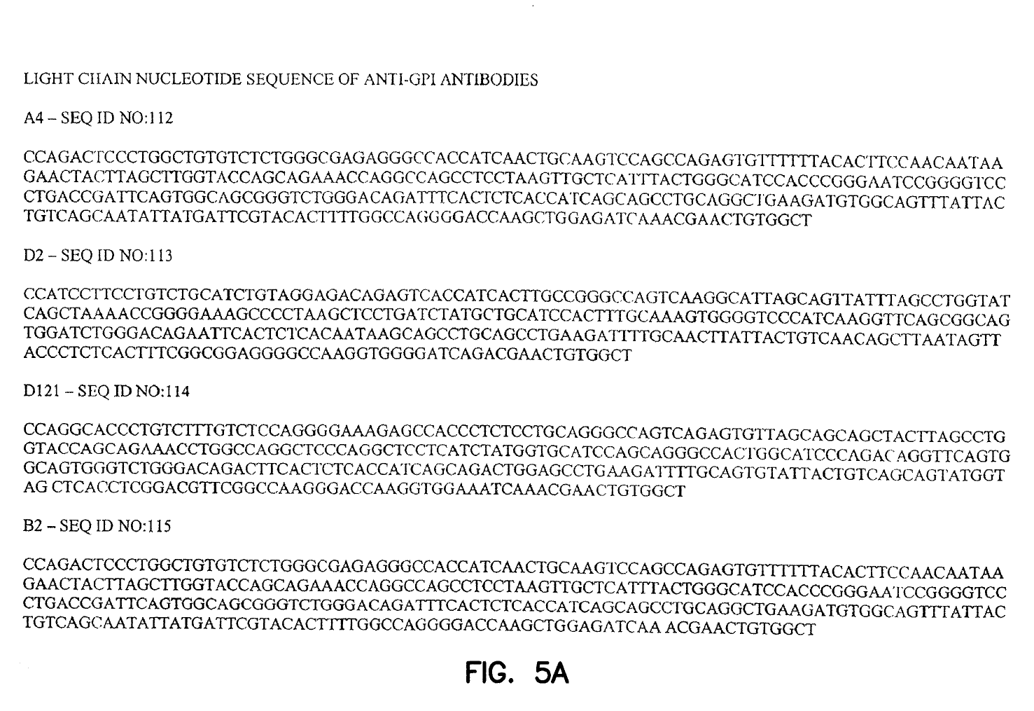

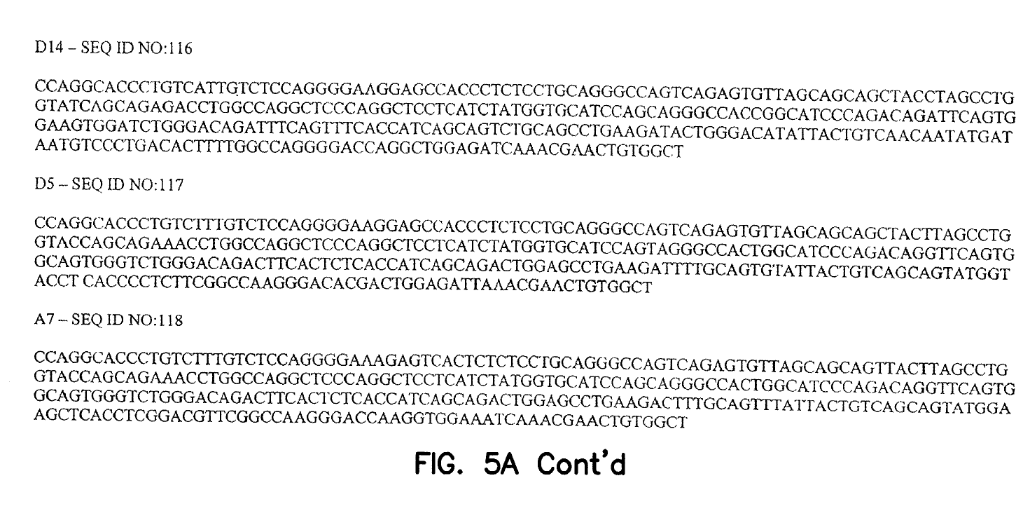

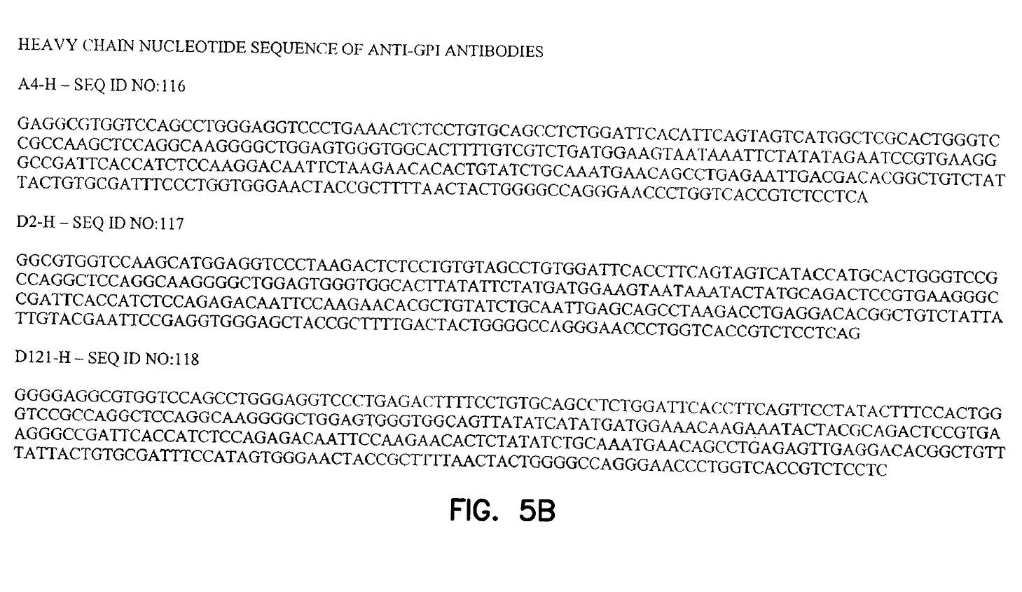

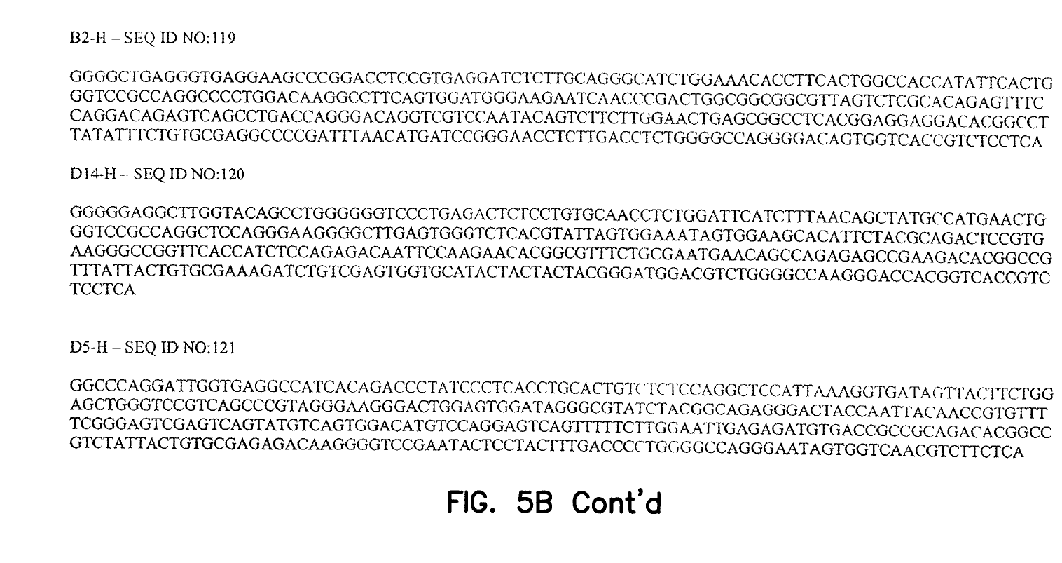

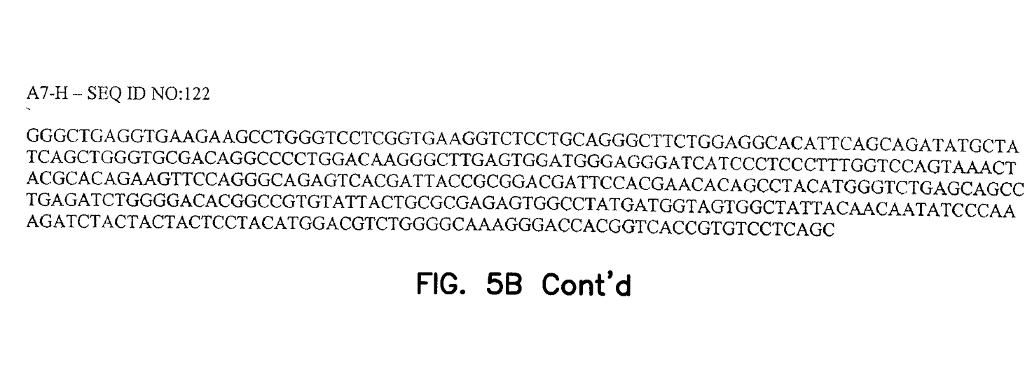

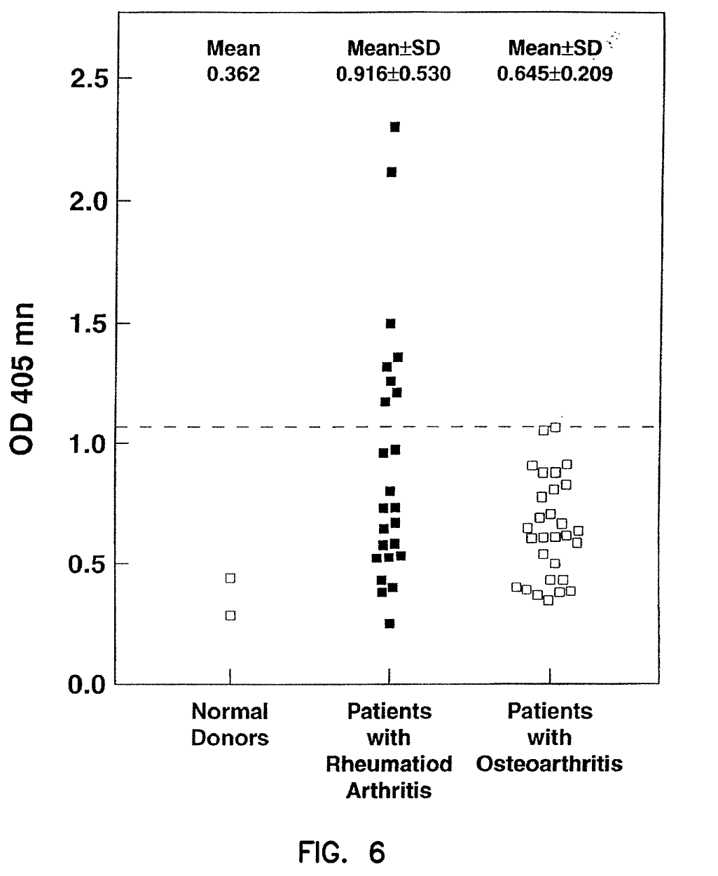

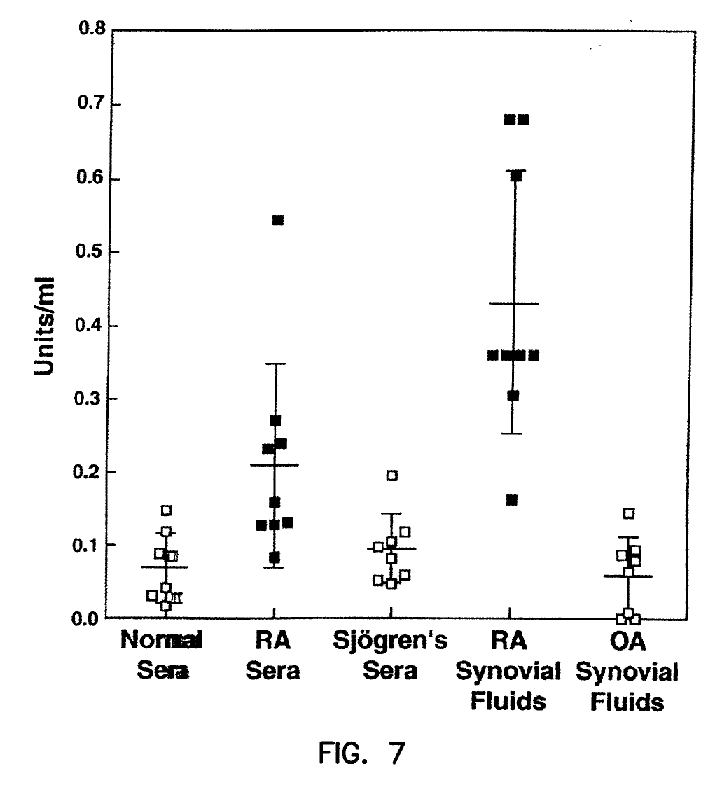

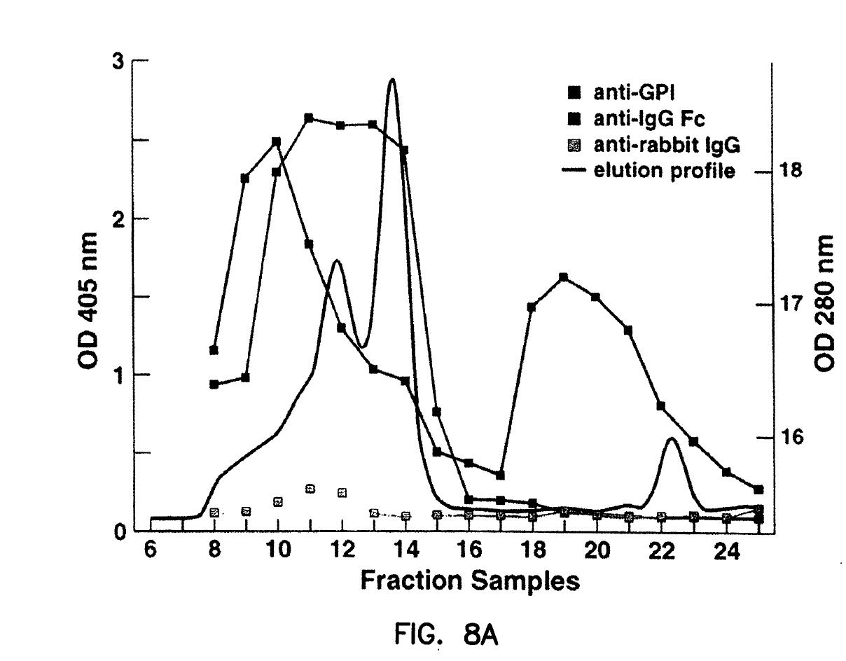

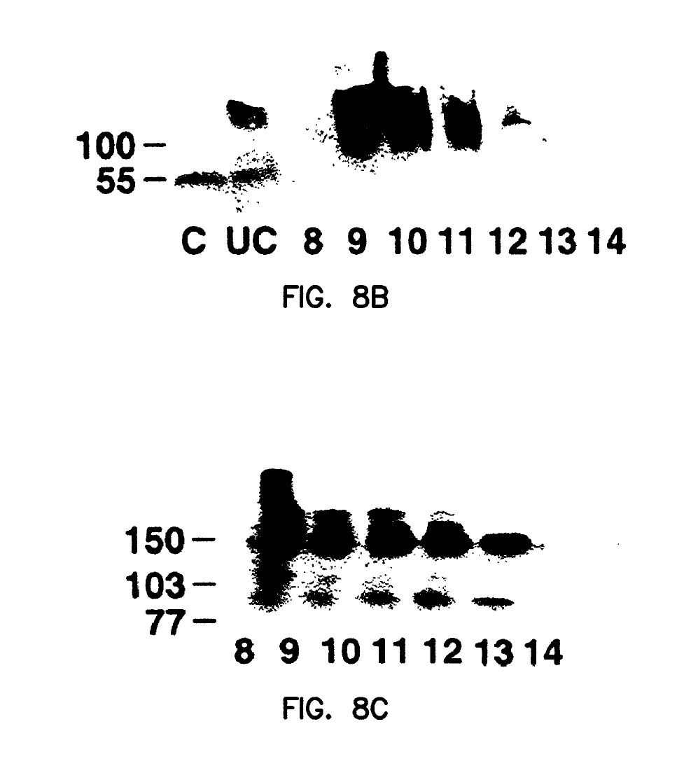

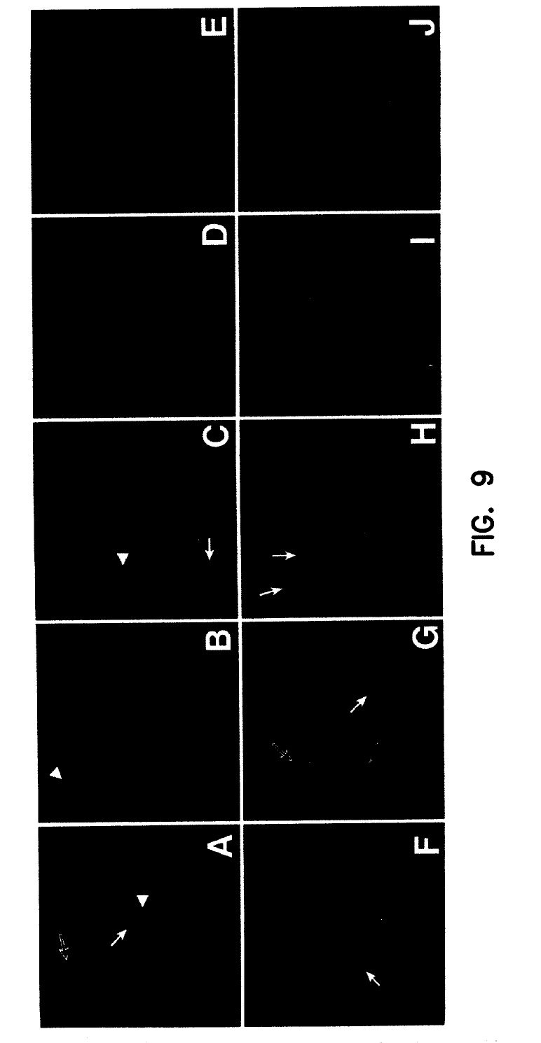

According to FIG. 3, the anti-GPI IgG antibodies cloned from a RA patient are highly somatically mutated and exhibit high replacement /silent (R/S) ratios. FIGS. 3AL and 3AH show the amino acid sequences of the light chain variable (VL) (3AL) and heavy chain variable (VH)(3AL) domains of the GPI specific Fabs. FIG. 3B shows a comparison of the heavy chain variable domains and the closest germline sequences. Dots indicate an amino acid identical in the anti-GPI Fab to the closest genuline. FR indicates framework region; CDR, indicates complementarity-determining region. (C). Comparison of the nucleotide and deduced amino acid sequences of the anti-GPI Fabs with the closest germline sequences demonstrates the frequency of silent (S) or replacement (R) mutations. In addition, the percent homology at nucleotide and amino acid levels to the closest germline are shown in FIG. 3C.

FIG. 4 shows the CDR and Framework sequences of the domains of FIG. 3.

FIGS. 5A and 5B show the light chain (A) and heavy chain (B) nucleotide sequences corresponding to the amino acid sequences of FIG. 3.

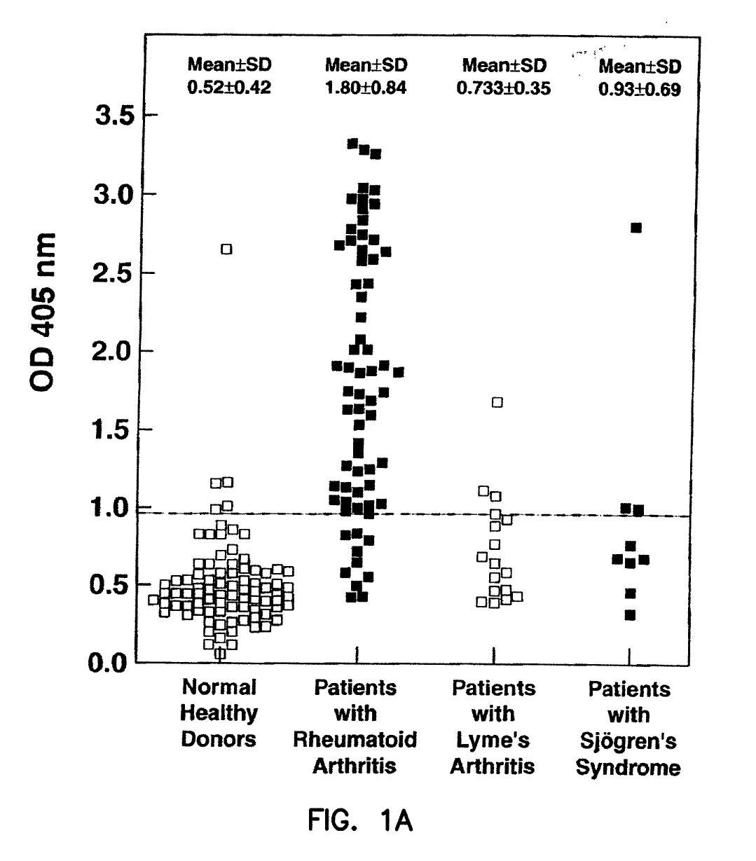

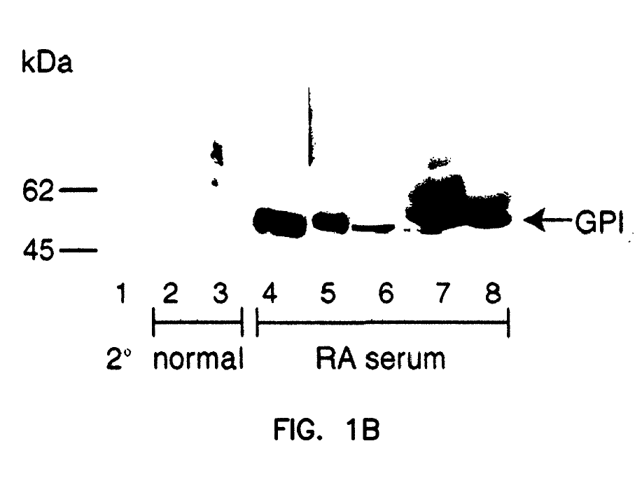

FIG. 6 shows a chart indicating the synovial fluid from patients with active RA contains IgG antibodies that bind specifically to GPI. Synovial fluid, diluted 1:200 in PBS, from 24 patients with active RA, 29 patients with osteoarthritis and 2 normal individuals were tested with the same ELISA procedure as shown in FIG. 1. Patient samples with OD 405 values of more than two standard deviations above the mean of the osteoarthritis synovial fluid values (>1.06) were considered positive.

FIG. 7 shows a chart of the GPI levels in RA patients.

According to FIG. 7, sera and synovial fluid of rheumatoid arthritis (RA) patients contain significantly increased concentrations of GPI. Eight sera from patients with RA, Sjögren's syndrome and normal healthy donors, respectively, were tested in a spectrophotometric assay measuring GPI enzymatic activity. In addition, eight synovial fluids from patients with active RA and osteoarthritis (OA) were tested in the same assay. Significantly higher levels of GPI were found in the RA sera compared to the Sjögren's sera (p<0.0001) and the normal sera (p<0.0001). In addition, significantly higher levels of GPI were also found in the RA synovial fluids compared to the OA synovial fluids (p<0.0001).

FIG. 8 shows a graph of the immune reaction products in synovial fluid of RA patients.

According to FIG. 8, elution profile (OD 280 nm) of synovial fluid from a patient with active RA arthritis separated by size exclusion chromatography (S-200) shows that GPI and anti-GPI antibodies were found as immune complexes as well as in free forms. The different elution fractions were coated on ELISA wells (A) and analyzed by Western blotting (B stained with a rabbit anti-human GPI antibody and C stained with an antihuman IgG Fc antibody). ELISA analysis revealed binding of the anti-GPI antibody corresponding to two peaks (A). The first peak corresponded to the first three fractions after the void volume and exhibited a molecular mass of 200 kDa and higher (B). The second GPI peak corresponded to free GPI with a molecular mass of approximately 55 kDa. The secondary anti-rabbit IgG did not bind to any of the fractions (A). Binding of anti-human IgG Fc antibody to the first fractions after the void volume was also observed. The first of these fractions contained complexes higher than 200 kDa, whereas the remaining predominantly contained free IgG (C). Unseparated synovial fluid, uncentrifuged (UC) and centrifuged (C), stained with the anti-GPI antibody (B) demonstrated that the immune complexes could be precipitated by high-speed centrifugation.

FIG. 9 shows microscope slides of the GPI profile in RA patients. According to FIG. 9, distribution of GPI in synovial tissue of patients with either active RA synoviitis (A-D and F-I) or osteoarthritis (E and J). Laser scanning confocal microscopy of frozen tissue section was stained with rabbit anti-GPI (green) and counter stained with propidium iodide (red). Intense staining in the RA synovial tissue was found corresponding to the endothelial surface of the synovial arterioles (A-C, arrowheads) and capillaries (C, arrow), but not of the venoles (A, open arrow). B is a magnification of a part of C. Intense patchy staining corresponding to the surface lining of the hyperplastic synovium (F-H), and particularly to the villous hypertrophy (G, open arrow), is shown. The staining of the synoviocytes in the villous and some other areas of the surface lining (G-H, closed arrows) were partly located intracellularly, whereas, in other areas the patchy staining appeared to be material precipitated on the surface lining (F, arrow). No staining of the RA synovial tissue was observed with the secondary FITC-labeled antirabbit IgG antibody alone (D and I). No intense staining was observed in the synovium from osteoarthritis patients (E and J) except for the faint diffuse cytoplasmic staining in most cells, most pronounced in smooth muscle cells, as also observed in the RA synovium.

### DEFINITIONS

Certain terms used to describe the present invention are defined in the section of the Detailed Description immediately preceding the Examples. Undefined terms have the ordinary, typical definitions provided in the art.

### DETAILED DESCRIPTION OF THE INVENTION

One of the models that has been designed recently to study autoimmune disease such as rheumatoid arthritis and related arthritic afflictions is the recently described K/BxN T-cell receptor (TCR) transgene RA model in which a destructive and chronic polyarthritis with no inflammation elsewhere is observed with high penetrance^{5;6} (these are cites to references given at the end of the Detailed Description). This mouse model shows many features in common with human RA including leukocyte invasion, synoviocyte proliferation, pannus formation, synovitis, and cartilage and bone erosion. It also shares immunological abnormalities with human RA including polyclonal B cell activation, hypergammaglobulinemia and, with the exception of rheumatoid factor, autoantibody production. The model was generated by crossing an autoimmunity-prone nonobese diabetic NOD mouse with a C57B1/6 mouse transgenic for a TCR recognizing a bovine ribonuclease (KRN) peptide presented by I-A^{k}. Study of this mouse model has revealed that glucose-6-phosphate isomerase (GPI), a glycolytic enzyme, can act as a self antigen and a target of arthritogenic IgG antibodies.

GPI is a cytosolic enzyme present in eukaryotes, bacteria, and archaea, that catalyzes the interconversion of D-glucose 6-phosphate and D-fiuctose 6-phosphate, an essential reaction of glycolysis and gluconeogenesis. In addition, GPI also has several roles outside the cell, where it has been observed to flnction as a cytokine and growth factor. Proteins initially described as neuroleukin (NLK)^{7;8}, autocrine motility factor (AMF)⁹, and differentiation and maturation mediator (DMM) 10 have been found to be identical to GPI. Neuroleukin is secreted by T cells and promotes the survival of some embryonic spinal neurons and sensory nerves. It also causes differentiation of B cells into mature antibody-secreting cells^{11;12}. AMF is a product of tumor cells that stimulates cancer cell migration and may be involved in cancer metastasis and invasion⁹. DMM was isolated from T cell culture media and shown to cause in vitro differentiation of human myeloid leukemia cells to terminal monocytic cells¹⁰.

While some have found the K/BxN TCR transgene RA mouse model interesting, there has been no evidence that GPI is an important autoantigen in the development of human autoimmune disease such as RA or related arthritides. In fact, researchers have recently cast doubt on the ability of such animal models to indicate features of human autoimmune disease. Several have criticized the animal models as not indicating the difficulties, etiologies, causes and symptoms of RA in humans. See R. Holmdahl, Arthritis Res., 2000; 2(3)175-8.

According to the invention, it has been discovered that human GPI is an antigen involved in human autoimmune disease. Studies concerning the human autoimmune response to human GPI have been conducted using serology and human antibody cloning by phage display. Patients with RA, but not healthy individuals, exhibit IgG antibodies against GPI and increased levels of soluble GPI. Human IgG anti-GPI antibodies have been cloned from a phage display library generated from the bone marrow of a patient with RA and are of high affinity for GPI. Sequence analyses indicate that the antibodies are generated in an antigen-driven response. GPI and anti-GPI antibodies are found in synovial fluids of patients with active RA at even higher levels than in the serum and form immune complexes. In RA synovium, increased levels of GPI are found on the surface of the arteriole endothelial cells and on the surface of the synovial lining.

The present invention is based upon above-described discovery that autoimmune diseases such as rheumatoid arthritis are at least in part the result of an immune reaction between human glucose-6-phosphate isomerase (GPI) and a set of human antibodies to GPI, termed anti-GPI antibodies (AGPI Ab). GPI is a normal constituent of living tissue. Usually, the human immune system does not contain antibodies that are reactive with GPI. As explained below, it is thought that triggering events may lead to the development of autoreactivity to GPI. Despite a lack of understanding of the etiology of this immune reaction, it has been found that intervention with the GPI-AGPI Ab immune reaction in humans ameliorates or arrests the autoimmune disease.

The present invention is directed to the therapeutic agents useful in diagnosing and treating autoimmune disease as well as the diagnostic and treatment methods themselves. The therapeutic agents as well as the methods are based upon the discovery that GPI is a human antigen that at least in part is involved in the development of human autoimmune disease such as rheumatoid arthritis.

### The AGPI Ab and Immunopolypeptides

The AGPI Ab and immunopolypeptides of the invention immunoreact with epitopal sites of GPI. GPI is the antigenic target of AGPI Ab and the immune complex that is formed is believed to trigger a cascade of immunological events leading to the symptoms of human autoimmune disease such as rheumatoid arthritis. Development of a regimen for arresting the production and/or reaction of native human AGPI Ab is believed to provide an especially effective treatment for human autoimmune disease such as rheumatoid arthritis.

The AGPI Ab is based upon the human immunoglobulin structure including IgA, IgE, IgG and IgM. Its preferable structure is IgG. Its forms of variable chain sequences are provided in FIG. 3A and by SEQ ID NO's 1-14. Also included are amino acid sequences that have significant homology with AGPI Ab, such as at least about 80% homology, preferably at least about 90% homology, more preferably at least about 95% homology, most preferably at least about 99% homology with the AGPI Ab sequences provided by the human immunoglobulin structure and SEQ ID NO's: 1-14. The isolated or purified form of the AGPI Ab is preferred and can be obtained by phage cloning as discussed below. The AGPI Ab and its substantially homologous sequences form the molecular basis for derivation of the other therapeutic agents of the invention.

The immunopolypeptide of the invention constitutes any polypeptide that binds to human GPI with a dissociation constant equal to, or less than, about 10⁻⁷. Preferably, the binding is specific for GPI with little or no contribution from non-specific binding. Especially preferably, the binding is immunobinding such that the immunopolypeptide does not bind with other enzymes of similar function. Preferably, the immunopolypeptide of the invention has an amino acid sequence that incorporates any of the CDR amino acid segments set forth in FIG. 4A, or an amino acid segment that has significant homology with the FIG. 4A CDR segments, such as at least about 80% homology, preferably at least about 90% homology, more preferably at least about 95% homology, most preferably at least about 99% homology with the CDR segments of FIG. 4A (hereinafter these ranges of homology are defined as significant homology, substantial homology, significantly homogeneous or as significant homologs). These CDR segments of FIG. 4A have amino acid sequences as set forth in SEQ ID NO's 15-56. In its most basic form, the immunopolypeptide is a single amino acid chain, which either incorporates any one or more of the CDR segments as set forth in SEQ ID NO's 15-56, has a segment having significant homology (as described above) with any one or more of these CDR segments, or is any one or more of the CDR segments. These CDR segments may be grouped into heavy chain CDR's having SEQ ID NO's 15-35 and the light chain CDR's having SEQ ID NO's 36-56. Especially preferably, the immunopolypeptide contains a triplet of these CDR sequences wherein each CDR is individually chosen from either or both of the light and heavy CDR groups. Preferably, the triplet of CDR sequences is chosen from one of the light and heavy CDR groups. More preferably, the triplet is chosen so that it matches the CDR's of a single chain of an Fab fragment of FIGS. 3AL and 3AH.

Preferably, the CDR's chosen for the immunopolypeptide are selected so as to bind to GPI. Preferably segments making the triplet of CDR's are appropriately spaced so as to provide a trifunctional-binding site. Preferably, the spaced triplet binds to GPI. Especially preferably, the trifunctional-binding site has spacer amino acid sequences between the CDR sequences that mimic the consensus number of amino acid units between CDR sequences of a human antibody. Although any spacer peptide sequence may be used, such as a short chain sequence of amino acid units having little or no ionic or lipophilic side chain properties, a preferred spacer peptide sequence is a human antibody variable region framework sequence. Human antibody variable region framework sequences are well known in the art, such as those in the National Center for Biotechnology Information (NCBI) genebank database. When the CDR triplets are combined with such a framework sequence, the immunopolypeptide mimics the variable region of a single chain of an AGPI Ab. Preferably, the human framework is a consensus human framework of a human immunoglobulin such as IgA, IgE, IgG and IgM, especially an IgG. More preferably, the framework has a sequence as given in FIG. 4B. These framework sequences have SEQ ID NO's 57-108. Most preferably, the immunopolypeptide incorporates a matched CDR and framework region of a single chain of an Fab fragment provided in FIG. 3AL and 3AH. Immunopolypeptide sequences with CDR and framework region amino acid sequences having significant homology to the Fab fragments of FIG. 3AL and 3AH are also preferred. Especially preferred are such immunopolypeptide sequences that strongly immunoreact with GPI. A strong immunoreaction is one having a dissociation constant equal to or less than about 10⁻⁷, preferably equal to or less than about 10⁻⁸, especially preferably equal to or less than about 10^{-9.}

The most basic structure of the immunopolypeptide is a single amino acid chain having the CDR selections or significant homologs thereof as described above. The immunopolypeptide also may have a structure that combines this single chain with a single chain of a constant region of a human immunoglobulin. In addition, the immunopolypeptide may be a combination of single chains. In particular, it may be a combination of any pair of single chains having CDR triplets or their significant homologs as defined above. Preferably, this combination includes the spacer amino acid units as discussed above. More preferably, this combination includes a triplet selected from the light CDR group and a triplet selected form the heavy CDR group. Especially more preferably, this combination includes the matched triplets and framework regions discussed above. Most preferably, this combination includes a light chain sequence and a heavy chain sequence with matched triplets and framework regions as discussed above. The preferred version of this most preferable combination is the variable region Fab or Fv fragment as provided by FIG. 3AL and 3AH. This preferred version may also be combined with the complete constant regions of an Fab or Fab' fragment of a human immunoglobulin to provide the complete Fab or Fab' fragment. The heavy chains of such complete Fab or Fab' fragments may be combined with a single heavy chain of an Fc fragment of an human immunoglobulin to provide at least one side of a complete antibody. With any of these double chain versions, the chains may be rearranged so that they are bound together in tandem fashion as a single chain. Any of these pairs may also be combined to provide a double pair combination, which will have a structure mimicking the “Y” form of a truncated or complete antibody.

When the immunopolypeptide has a structure like the variable region of an antibody (i.e. a CDR triplet spaced with an antibody framework sequence), it mimics, or in certain versions is, the variable region single chain of an Fab monoclonal antibody fragment. If the appropriate constant region sequence of an Fab fragment is added, the immunopolypeptide has a structure mimicking, or is, a complete single chain of an Fab or Fab' antibody fragment. If the CDR triplets are chosen from the light and heavy groups as discussed above, immunopolypeptide is a variable region single chain of a Fab monoclonal antibody fragment. With the addition of the appropriate constant region sequences from an Fab, an Fab' and/or an Fc fragment to this single light or heavy variable region chain, the immunopolypeptide is a full length heavy or light single chain of a monoclonal antibody. The immunopolypeptide may also be a combination of two such single chains of any of the foregoing descriptions. This combination may be two light chains, two heavy chains, two mixed CDR chains, or preferably a light and heavy chain combination. When the last combination includes the variable region and the optional constant region, it has a construction like that of an Fab or Fab' fragment. When this combination provides an Fab' fragment and two of such fragments are combined, the resulting immunopolypeptide is an F(ab')₂ monoclonal antibody fragment. When the immunopolypeptide is an F(ab)ₓ fragment plus a constant region Fc of a human immunoglobulin, it is a complete human monoclonal antibody. In its state as a complete antibody, its Fc region may also be modified to destroy or alter the complement binding sites or membrane Fc binding sites. The alterations or destruction of these binding sites of the Fc region may be accomplished by site directed mutagenesis.

Preferred species of the immunopolypeptide of the invention include the Fab variable region amino acid sequences provided in FIG. 3AL and 3AH. These sequences have SEQ ID NO's: 1-14. Also preferred are the Variants of these Fab fragments as well as amino acid sequences that have significant homology with these SEQ ID NO's: 1-14. Preferred CDR sequences for the immunopolypeptide of the invention include the amino acid sequences designated in the CDR columns of FIG. 4A. These CDR's have SEQ ID NO's: 15-56. Preferred framework sequences for the immunopolypeptide of the invention include the amino acid sequences designated in the framework columns of FIG. 4B. These framework sequences have SEQ ID NO's: 57-108. Preferred Fab' constant region sequences for the immunopolypeptide of the invention, which provide the heavy and light chain constant regions of Fab fragments, include those human consensus regions provided within the genebank of the National Center for Biotechnology Information.

### The Polynucleotide Encoding the Immunopolypeptide

The polynucleotide of the invention is produced by manipulation of the DNA sequences obtained from the phage library as discussed below. Recombinant techniques for obtaining the DNA encoding the source antibodies that immunoreact with GPI provide the DNA sequences encoding the matched Fab sequences of FIGS. 3AL and 3AH. These nucleotide sequences are given in FIG. 5A and 5B and have SEQ ID NO's: 109-122.

Using known techniques such as solid phase synthesis of oligonucleotides (See Beaucage and Caruthers, J Am Chem Soc., 24, 3184-3191 (1981), Efimov et al. Nucleic Acids Res., 13, 3651 (1985) and U.S. Pat. No. 5,464,759) or endonuclease digestion and recombinant DNA technology, or site directed mutagenesis, nucleotide sequences encoding the CDR's and spacer amino acid sequences and their significant homologs (i.e. the immunopolypeptide of the invention) may be produced. Religation of these CDR and spacer nucleotide segments using techniques known in the art will produce the polynucleotide encoding the immunopolypeptide of the invention. Additionally, nucleotide sequences for the consensus constant regions may be obtained from the genebank and used in known ligation procedures to engineer additive DNA sequences encoding still other forms of the immunopolypeptide described above, such as but not limited to the complete antibody, Fab' fragments, Fd fragments, complete single chains, single fused chains, as well as Fab and Fv fragments containing consensus constant regions. See the Cold Spring Harbor Laboratory Manuals cited below for the details involved in DNA sequence engineering.

Amino acid sequences of the invention may also be produced through synthetic methods well known in the art (Merrifield, Science, 85:2149 (1963)).

### Process For Preparation of the Immunopolyeptide

The CDR and spacer or framework sequences for the immunopolypeptide of the invention as well as the sequences for the AGPI Ab preferably are derived by known techniques from the mononuclear cells of a human patient having an autoimmune disease such as rheumatoid arthritis. These techniques and development of CDR sequences from antibodies is described, for example, in Antibodies, A Laboratory Manual by Harlow and Lane, Cold Spring Harbor Laboratory Press, Cold Spring Harbor, N.Y., 1988, and in Molecular Cloning, A Laboratory Manual by Sambrook, et al., Cold Spring Harbor Laboratory Press, Cold Spring Harbor, N.Y. 1989, the disclosures of which are incorporated herein by reference. The CDR development technique using a human source was the procedure used to provide the CDR sequences of FIG. 4A.

The CDR development technique first involves collection of immune cells sensitized to the specified antigen. In particular, for the CDR's of the invention, mononuclear cells from a patient afflicted with rheumatoid arthritis are collected. Preferably, these cells are from the bone marrow of the afflicted human patient. B cells and/or plasma cells from the spleen may also be harvested if appropriate. Human tissues such as peripheral blood, synovial tissue or immune tissues from non-human experimental animals genetically transformed to exhibit human immune cells may also be used as a source. The mononuclear cells are processed according to the phage display technology described by Barbas et al. Proc. Natl. Acad. Sci., USA 88, 7978-7982 (1991); and in U.S. Pat. Nos. 5,580,717; 5,972,656; 6,113,898; and 6,140,470; the disclosures of which are incorporated herein by reference. Briefly, either total RNA or RNA that has been processed to obtain the polyA-RNA is obtained from cells. After hybridization of an oligo-d(T) primer, the RNA (MRNA) is reverse transcribed to yield the corresponding cDNA. This cDNA provides the stock DNA coding material leading to development of the immunopolypeptide sequences of the invention. The mRNA or the cDNA may be amplified by PCR techniques to provide full length genes or through the use of selected primers to provide antibody fragments such as Fab, F(ab')₂, V_{H}, V_{L}, scFv, the complete partial constant region for the Fab, and the like. The cDNA or PCR products may then be inserted into a vector, such as a bacteriophage or a phagemid though use of recombinant DNA techniques well known in the art. Sambrook et al., Molecular Cloning, A Laboratory Manual, cited supra (1989). The vectors containing the cDNA or PCR products may then be transformed into bacteria to produce a library.

This procedure will provide a vector for transfection of bacteria and allow expression of the desired cDNA or PCR product, such as an antibody, single variable chains, Fab fragments. The procedure also allows for the production of a polypeptide, which is fused to a coat protein.

A semisynthetic library or naive library may also be developed by site directed mutagenesis of individual B cells. The resulting library if differentiated B cells may then be treated according to the following description of recombinant phage selection to select those B cells expressing membrane proteins that bind with GPI. See www.44.ncbi.nlm/nih.giv/pubmed/semisynthetic for the details of the development of the semisynthetic library of differentiated B cells; the disclosure of this reference is incorporated herein by reference.

The library of recombinant phage (or library of differentiated B cells) may be panned as described in the foregoing references and patents to select those phage carrying antibody chains that will bind with the antigen, GPI. The panning may be accomplished by combining the phage library with immobilized glycoprotein or protein, removing the phage not bound, followed by removing the bound phage. For efficient recombination, panning and bacterial transfection, the MRNA or cDNA stock material may be amplified using selected primers to provide antibody variable regions. The DNA encoding constant regions may be recombined in appropriate orientation once the desired expression vector is obtained.

The host bacterial cells such as E. coli or other suitable bacteria are transformed with the panned phage library to provide a library of transformed cells. The cells are separated to colonies carrying only single antibody genes by plating onto culture medium. The phage may also carry a selection marker such as an antibiotic resistance gene. Selection with culture medium carrying the selection marker provides cultures of bacteria that have been transfected. Examination of single cell cultures from single colonies by a binding assay using the GPI identifies those cultures exhibiting specific immunoreactivity.

Following selection of bacterial cultures exhibiting expression of polypeptide having specific immunoreactivity, the nucleotide sequences encoding CDR's, framework, single chain variable regions or single chain variable and constant regions of Fab, Fab', Fd, Fv, single fused chain and other fragments as described above may be conveniently identified by known procedures for nucleotide sequence identification.

In particular, the nucleotide sequences encoding the Fab sequences provided in FIG. 3AL and 3AH are determined by this technique. These nucleotide sequences are provided in FIG. 5A and 5B. The cultures providing expression of the desired polypeptides may also be manipulated by known recombinant techniques to insert into a vector (e.g. the recombinant phage) the nucleotide sequences for remainder of the desired immunopolypeptide amino acid sequence. Such sequences include, for example, the constant antibody regions of light and heavy chains as well as the Fc chain. Alternatively, the CDR DNA sequences obtained through sequencing of the phage or phagemid DNA, or the semisynthetic DNA sequences for significant homologs of the CDR DNA sequences prepared as described above, may be cloned into a vector carrying the DNA sequences encoding the spacers, framework and constant regions of the immunopolypeptide of the invention. Those DNA sequences are consensus sequences, are known and are available from gene bank sources as described above. Site directed mutagenesis may also be employed to provide Variants. If the phage library is designed to carry the nucleotide sequences for the antibody constant regions as well as the variable regions, those constant region DNA sequences may be used instead. Similarly, the framework DNA sequences obtained by sequence identification of the phage DNA from the immunoreactive bacterial cultures may be used as the nucleotide sequences encoding the framework amino acid sequences of the immunopolypeptides of the invention.

In addition to use of bacterial host cells for expression of the immunopolypeptide of the invention, mammalian host cells such as chinese hamster ovary cells may also be used. The nucleotide sequence encoding the desired immunopolypeptide obtained as described above may be inserted into an expression cassette for mammalian host cells. Transfection and expression of the nucleotide sequence in the mammalian host cells will produce the immunopolypeptide. These recombinant cells are capable of expressing the appropriately folded, complete monoclonal antibody.

Combinations of chains such as chains for an Fab fragment or light and heavy variable region chains can also be expressed by a single cell following the techniques given in the foregoing references and patents. Mixing the nucleotide sequences for the selected immunospecific light and heavy chains and insertion into a phage followed by bacterial transfection will provide both chains. The techniques described above may be followed to provide antibody fragments or full length antibodies. Alternatively, single chain expression products may be mixed at appropriate ratios and coupled by disulfide ligation to provide two chain combinations.

The immune cells from a source such as an experimental non-human mammal treated with the GPI antigen (see the following discussion) or a patient afflicted by rheumatoid arthritis may also be fused with immortalized cells to provide hybridomas expressing the library of antibodies derived from the patient. The techniques described above and in the Cold Spring Harbor Laboratory Manuals cited above provide the protocols for obtaining monoclonal antibodies from hybridomas.

Single chains typically are produced by the bacterial cell culture techniques described above. The three dimensional structure of a typical antibody is known to be highly stable and reconstitutable. Consequently, under appropriate conditions known in the art, these single chains may be ligated and folded to provide active antibody configurations. Ligation may be achieved by conducting in vitro disulfide bond formation. Proper folding may be accomplished by dilute constitution in aqueous physiological media. Protein folding and disulfide ligation techniques are well known in the art.

The following detailed procedure provides further explanation for production of the immunopolypeptide of the invention.

PCR amplification of Fd and K regions from the MRNA of the source mononuclear cells a may be performed as described by Sastry et al., Proc. Natl. Acad. Sci U.S.A., 86, 5728 (1989). The PCR amplification is performed with cDNA obtained by the reverse transcription of the mRNA with primer specific for amplification of heavy chain sequences or light chain sequences.

The PCR amplification of messenger RNA (mRNA) isolated from the mononuclear cells with oligonucleotides that incorporate restriction sites into the ends of the amplified product may be used to clone and express heavy chain sequences (e.g., the amplification of the Fd fragment) and K light chain sequences from mouse spleen cells. The oligonucleotide primers, which are analogous to those that have been successfully used for amplification of V_{H} and V_{L} sequences (see Sastry et al., Proc. Natl. Acad. Sci U.S.A., 86, 5728 (1989)), may be used for these amplifications. Restriction endonuclease recognition sequences are typically incorporated into these primers to allow for the cloning of the amplified fragment into a suitable vector (i.e. a phagemid or a A phage) in a predetermined reading frame for expression.

Phage assembly proceeds via an extrusion-like process through the bacterial membrane. For example filamentous phage M13 may be used for this process. This phage has a 406-residue minor phage coat protein (cpIII) which is expressed before extrusion and which accumulates on the inner membrane facing into the periplasm of E. coli. The two functional properties of cpIII, infectivity and normal (nonpolyphage) morphogenesis have been assigned to roughly the first and second half of the gene. The N-terminal domain of cpIII binds to the F' pili, allowing for infection of E. coli, whereas the membrane-bound C-terminal domain, P198-S406, serves the morphogenic role of capping the trailing end of the filament according to the vectorial polymerization model.

A phagemid vector may be constructed to fuse the antibody fragment chain such as an Fab, Fab' or preferably an Fd chain with the C-terminal domain of cpIII (see Barbas et al., Proc. Natl. Acad. Sci. USA, 88, 7978 (1991)). A flexible five-amino acid tether (GGGGS), which lacks an ordered secondary structure, may be juxtaposed between the expressed fragment chain and cpIII domains to minimize interaction. The phagemid vector may also be constructed to include a nucleotide coding for the light chain of a Fab fragment. The cpIII/Fd fragment fusion protein and the light chain protein may be placed under control of separate lac promoter/operator sequences and directed to the periplasmic space by pelB leader sequences for functional assembly on the membrane. Inclusion of the phage F1 intergenic region in the vector allows for packaging of single-stranded phagemid with the aid of helper phage. The use of helper phage superinfection may result in expression of two forms of cpIII. Consequently, normal phage morphogenesis may be perturbed by competition between the cpIII/Fd fragment fusion protein and the native cpII of the helper phage for incorporation into the virion. The resulting packaged phagemid may carry native cpIII, which is necessary for infection, and the fusion protein including the Fab fragment, which may be displayed for interaction with an antigen and used for selection. Fusion at the C-terminal domain of cpIII is necessitated by the phagemid approach because fusion with the infective N-terminal domain would render the host cell resistant to infection. The result is a phage-displaying antibody combining sites (“Phabs”). The antibody combining sites, such as Fab fragments, are displayed on the phage coat. This technique may be used to produce Phabs which display recombinantly produced Fab fragments, such as recombinantly produced Fab fragments that immunoreact with a antigen, on the phage coat of a filamentous phage such as M13.

A phagemid vector (i.e. pComb 3 or pComb3H) which allows the display of antibody Fab fragments on the surface of filamentous phage, has been described (see Barbas et al., Proc. Natl. Acad. Sci. USA, 88, 7978 (1991). Xho I and Spe I sites for cloning PCR-amplified heavy-chain Fd sequences are included in pComb 3 and pComb 3H. Sac I and Xba I sites are also provided for cloning PCR-amplified antibody light chains. These cloning sites are compatible with known mouse and human PCR primers (see, e.g., Huse et al., Science, 246, 1275-1281 (1989)). The nucleotide sequences of the pelB leader sequences are recruited from the λ, HC2 and λ LC2 constructs described in Huse et al, ibid, with reading frames maintained. Digestion of pComb 3 and pComb 3H, encoding a selected Fab, with Spe I and Nhe I permit the removal of the gene III fragment, which includes the nucleotide sequences coding for the antibody Fab fragments. Because Spe I and Nhe I produce compatible cohesive ends, the digested vector may also be religated to yield a phagemid that produces soluble Fab.

Phabs may be produced by overnight infection of phagemid containing cells (e.g., infected E. coli XL-1 Blue) yielding typical titers of 10¹¹ cfu/ml. By using phagemids encoding different antibiotic resistances, ratios of clonally distinct phage may easily be determined by titering on selective plates. In single-pass enrichment experiments, clonally mixed phage may be incubated with an antigen-coated plate. Nonspecific phage will be removed by washing, and bound phage may then be eluted with acid and isolated.

### Anti-idiotypic Antibody to AGPI Ab

The invention also includes an anti-idiotypic antibody to AGPI Ab. This antiidiotypic antibody is therapeutically useful for amelioration of the symptoms of rheumatoid arthritis. Preferably, the anti-idiotypic antibody is a chimeric monoclonal antibody, especially preferably a humanized chimeric monoclonal antibody, most preferably a human anti-idiotypic monoclonal antibody produced for example by immunization of a transgenic animal carrying human immune cells.

The anti-idiotypic antibody is prepared according to techniques known in the art. For example, it may be prepared by hybridoma or phage—microbe expression according to the procedures outlined in Antibodies, A Laboratory Manual by Harlow and Lane, Cold Spring Harbor Laboratory Press, Cold Spring Harbor, N.Y., 1988, and in Molecular Cloning, A Laboratory Manual by Sambrook, et al., Cold Spring Harbor Laboratory Press, Cold Spring Harbor, N.Y. 1989, the disclosures of which are incorporated herein by reference.

In summary, a laboratory mammal such as a mouse or rat, or a transgenic animal carrying the human immune system as discussed below, may be treated with the AGPI Ab, preferably an Fab or similar fragment of AGPI Ab, and appropriate adjuvants to develop an immune response to the protein. The mononuclear cells from the spleen, bone marrow or other appropriate source are extracted and processed according to a hybridoma or phage technique to produce a library of cells expressing antibodies.

The library is then screened to identify those cells and/or cellular material that immunoreact with AGPI Ab. The resulting sublibrary is then preferably further screened to eliminate cells and/or cellular material that also cross-react with the Fc fragment of human IgG and with somatic IgG. The resulting preferred sublibrary then constitutes non-human anti-idiotypic antibodies that immunoreact with the variable region of human AGPI Ab. The sublibrary antibodies are sequenced and the amino acid sequences of the CDR segments determined. The corresponding CDR nucleotide sequences are then cloned into a human immunoglobulin nucleotide sequence, preferably an IgG nucleotide sequence, so that when the resulting nucleotide sequence is expressed, the cloned CDR sequences are positioned in appropriate locations within the IgG framework region. This procedure follows the techniques for development of humanized chimeric antibodies as given EPO Publication No. 0239400, (Jones et al., Nature, 321, 522-525 (1986) (Verhoeyen et al., Science, 239, 1534-1536 (1988); Riechmann et al., Nature, 332, 323-327 (1988); and U.S. Pat. No. 6,180,370, the disclosures of which are incorporated herein by reference.

In particular, the humanized, chimeric monoclonal anti-idiotypic antibody may be produced by expressing recombinant DNA segments encoding the heavy and light chain CDR's from a donor monoclonal antibody capable of binding to the desired antigen, namely the variable region, and preferably, the hypervariable variable sequences of human AGPI Ab. Exemplary DNA sequences designed in accordance with the present invention code for the polypeptide chains comprising heavy and light chain CDR's with substantially human framework regions. Due to codon degeneracy and non-critical amino acid substitutions, other DNA sequences can be readily substituted for those sequences.

The DNA segments will typically further include an expression control DNA sequence operably linked to the humanized monoclonal antibody coding sequences, including naturally-associated or heterologous promoter regions. Preferably, the expression control sequences will be eukaryotic promoter systems in vectors capable of transforming or transfecting eukaryotic host cells, but control sequences for prokaryotic hosts may also be used. Once the vector has been incorporated into the appropriate host, the host is maintained under conditions suitable for high level expression of the nucleotide sequences, and, as desired, the collection and purification of the humanized light chains, heavy chains, light/heavy chain dimers or intact antibodies, binding fragments or other monoclonal antibody forms may follow (see, S. Beychok, Cells of Monoclonal antibody Synthesis, Academic Press, New York, (1979), which is incorporated herein by reference).

Human constant region DNA sequences can be isolated in accordance with well known procedures from a variety of human cells, but preferably immortalized B-cells (see, Kabat op. cit. and WO 87/02671). The CDR's for producing the monoclonal antibodies of the present invention will be similarly derived from monoclonal antibodies capable of binding to the variable region of AGPI Ab and produced by well known methods in any convenient mammalian source including, mice, rats, rabbits, or other vertebrates, capable of producing antibodies. Suitable source cells for the constant region and framework DNA sequences, and host cells for monoclonal antibody expression and secretion, can be obtained from a number of sources, such as the American Type Culture Collection (“Catalogue of Cell Lines and Hybridomas,” sixth edition (1988) Rockville, Md., U.S.A., which is incorporated herein by reference).

In addition to the humanized monoclonal antibodies specifically described herein, other “substantially homologous” modified monoclonal antibodies to the native sequences can be readily designed and manufactured utilizing various recombinant DNA techniques well known to those skilled in the art. For example, the framework regions can vary specifically from the sequences of consensus human frameworks at the primary structure level by several amino acid substitutions, terminal and intermediate additions and deletions, and the like. Moreover, a variety of different human framework regions may be used singly or in combination as a basis for the humanized monoclonal antibodies of the present invention. In general, modifications of the genes may be readily accomplished by a variety of well-known techniques, such as site-directed mutagenesis (see, Gillman and Smith, Gene, 8, 81-97 (1979) and S. Roberts et al., Nature, 328, 731-734 (1987), both of which are incorporated herein by reference).

A further technique for production of the anti-idiotypic monoclonal antibody of the invention involves the use of an experimental transgenic animal carrying a human immune system. Following the immunization, hybridization or phage production and selection techniques described above, a library of hybridomas or phage can be produced which express fully human anti-idiotypic monoclonal antibodies. Although the CDR and framework regions of these antibodies may be human sequences, there is no need to humanize the remaining portions of these antibodies. Their sequences are already human owing to their human immune system origin.

Alternatively, second immunopolypeptides comprising only a portion of the antiidiotypic antibody (to the AGPI Ab) may be produced. These second immunopolypeptides possess one or more anti-idiotypic antibody activities (e.g., immunobinding activity with AGPI Ab) and have all of the structural variations described above for the immunopolypeptides of the invention. These second immunopolypeptide fragments may be produced by proteolytic cleavage of intact antibodies by methods well known in the art, or by inserting stop codons at the desired locations in the vectors encoding the anti-idiotypic monoclonal antibody or by following the procedures given above for the immunopolypeptides of the invention. They may also be produced by use of site-directed mutagenesis, such as after CHI to produce Fab fragments or after the hinge region to produce (Fab')2 fragments. Single chain antibodies may be produced by joining VL and VH with a DNA linker (see, Huston et al., op. cit., and Bird et al., op. cit.). The nucleic acid sequences of the present invention capable of ultimately expressing the desired humanized antibodies can be formed from a variety of different polynucleotides (genomic or cDNA, RNA, synthetic oligonucleotides, etc.) and components (e.g., V, J, D, and C regions), as well as by a variety of different techniques. Joining appropriate synthetic and genomic sequences is presently the most common method of production, but cDNA sequences may also be utilized (see, European Pat. Publication No. 0239400 and L. Reichmann et al., Nature, 332, 323-327 (1988), both of which are incorporated herein by reference).

### Host Cells

As stated previously, the DNA sequences for the immunopolypeptide, anti-GPI Ab, anti-idiotypic antibody or second immunopolypeptide may be expressed in hosts after the sequences have been operably linked to (i.e., positioned to ensure the functioning of) an expression control sequence. These expression vectors are typically replicable in the host organisms either as episomes or as an integral part of the host chromosomal DNA. Commonly, expression vectors will contain selection markers, e.g., tetracycline or neomycin, to permit detection of those cells transformed with the desired DNA sequences (see, e.g., U.S. Pat. No. 4,704,362, which is incorporated herein by reference).

E. coli is one prokaryotic host useful particularly for cloning the DNA sequences of the present invention. Other microbial hosts suitable for use include bacilli, such as Bacillus subtilus, and other enterobacteriaceae, such as Salmonella, Serratia, and various Pseudomonas species. In these prokaryotic hosts, one can also make expression vectors, which will typically contain expression control sequences compatible with the host cell (e.g., an origin of replication). In addition, any number of a variety of well known promoters will be present, such as the lactose promoter system, a tryptophan (trp) promoter system, a beta-lactamase promoter system, or a promoter system from phage lambda. The promoters will typically control expression, optionally with an operator sequence, and have ribosome binding site sequences and the like, for initiating and completing transcription and translation.

Other microbes, such as yeast, may also be used for expression. Saccharomyces is a preferred host, with suitable vectors having expression control sequences, such as promoters, including 3-phosphoglycerate kinase or other glycolytic enzymes, and an origin of replication, termination sequences and the like as desired.

In addition to microorganisms, mammalian tissue cell culture may also be used to express and produce the polypeptides of the present invention (see, Winnacker, “From Genes to Clones,” VCH Publishers, New York, N.Y. (1987), which is incorporated herein by reference). Eukaryotic cells are actually preferred, because a number of suitable host cell lines capable of secreting intact monoclonal antibodies have been developed in the art, and include the CHO cell lines, various COS cell lines, HeLa cells, preferably myeloma cell lines, etc, and transformed B-cells or hybridomas. Expression vectors for these cells can include expression control sequences, such as an origin of replication, a promoter, an enhancer (Queen et al., Immunol. Rev., 89, 49-68 (1986), which is incorporated herein by reference), and necessary processing information sites, such as ribosome binding sites, RNA splice sites, polyadenylation sites, and transcriptional terminator sequences. Preferred expression control sequences are promoters derived from monoclonal antibody genes, SV40, Adenovirus, cytomegalovirus, Bovine Papilloma Virus, and the like. The vectors containing the DNA segments of interest (e.g., the heavy and light chain encoding sequences and expression control sequences) can be transferred into the host cell by well-known methods, which vary depending on the type of cellular host. For example, calcium chloride transfection is commonly utilized for prokaryotic cells, whereas calcium phosphate treatment or electroporation may be used for other cellular hosts. (See, generally, Maniatis et al., Molecular Cloning: A Laboratory Manual, Cold Spring Harbor Press, (1982), which is incorporated herein by reference.) Once expressed, the AGPI Ab, immunopolypeptide, anti-idiotypic antibody and/of second immunopolypeptide as well as their dimers, individual light and heavy chains, or other forms of the present invention, can be isolated and purified according to standard procedures of the art, including ammonium sulfate precipitation, affinity columns, column chromatography, gel electrophoresis and the like (see, generally, R. Scopes, “Protein Purification”, Springer-Verlag, N.Y. (1982)). (See also, generally, Immunological Methods, Vols. I and II, Lefkovits and Pernis, eds., Academic Press, New York, N.Y. (1979 and 1981)).

### Antisense Oligonucleotides

The antisense oligonucleotides of the invention are designed to bind as complementary sequences with either the DNA or the MRNA encoding the AGPI Ab or GPI itself. When the antisense oligonucleotide sequence is directed to the AGPI Ab nucleotide, this hybridization interrupts the expression of the in vivo AGPI Ab thus ameliorating the immune reaction leading to autoimmune diseases such as rheumatoid arthritis. When the antisense oligonucleotide sequence is directed to the GPI nucleotide, the amount of oligonucleotide administered is titrated so as to determine the appropriate dosage needed to bring the GPI concentration in body tissue such as synovial fluid to a normal level.

The antisense oligonucleotides of the invention may range in length from about 4 to about 100 bases in length, preferably from about 10 to about 50, more preferably from about 10 to 30. They may incorporate the natural complementary bases relative to the DNA or MRNA with which they are to hybridize, or they may incorporate one or more non-natural modifications to lengthen their in situ half lives. Those modifications include such changes as use of thiophosphate groups, use of alkylated base side chains as well as others. The techniques for design of antisense oligonucleotides, their modification and their synthesis are provided in U.S. Pat. Nos. 6,184,211; 6,166,197 and 6,150,510, the disclosures of which are incorporated herein by reference.

In one aspect of the embodiment of invention the oligonucleotides have a plurality of monomeric sub-units or nucleobases. Nucleobases according to the invention include purines and pyrimidines such as adenine, guanine, cytosine, uridine, and thymine, as well as other synthetic and natural nucleobases such as xanthine, hypoxanthine, 2-aminoadenine, 6-methyl and other alkyl derivatives of adenine and guanine, 2-propyl and other alkyl derivatives of adenine and guanine, 5-halo uracil and cytosine, 6-azo uracil, cytosine and thymine, 5-uracil (pseudo uracil), 4-thiouracil, 8-halo, amino, thiol, thioalkyl, hydroxyl and other 8-substituted adenines and guanines, 5-trifluoromethyl and other 5-substituted uracils and cytosines, 7-methylguanine. Further purines and pyrimidines include those disclosed in U.S. Pat. No. 3,687,808, those disclosed in the Concise Encyclopedia of Polymer Science And Engineering, pages 858-859, Kroschwitz, J. I., ed. John Wiley & Sons, 1990, and those disclosed by Englisch, et al., Angewandte Chemie, International Edition 1991, 30, 613.

The nucleotide bases may be sequentially linked to form the oligonucleotides of the invention using standard oligonucleotide synthesis protocols. An oligo-nucleotide for the purposes of the present invention is a backbone oligomer having at least two nucleotides covalently bound by a phosphate linkage. An oligo-nucleotide can have a plurality of nucleotides coupled through phosphorous containing or modified phosphorous containing linkages.

Methods of coupling monomeric nucleotides according to the invention include solution phase and solid phase chemistries. Representative solution phase techniques are described in U.S. Pat. No. 5,210,264. Representative solid phase techniques are those typically employed for DNA and RNA synthesis utilizing standard phosphoramidite chemistry. (see, e.g., Protocols For Oligonucleotides And Analogs, Agrawal, S., ed., Humana Press, Totowa, N.J., 1993.) A preferred synthetic solid phase synthesis utilizes phosphoramidites as activated phosphates. The intermediate phosphite compounds are subsequently oxidized using known methods. This allows for synthesis of linkages including phosphodiester or phosphorothioate phosphate linkages depending upon oxidation conditions selected. Other phosphate linkages can also be generated. These include phosphorodithioates, phosphotriesters, alkyl phosphonates, phosphoroselenates and phosphoramidates.

For the purposes of the invention, the nucleotide monomeric units may also be substituted with alkyl groups. Such substitutions have been found to increase the in vivo half life of antisense oligonucleotides. The alkyl groups include but are not limited to substituted and unsubstituted straight chain, branch chain, and alicyclic hydrocarbons, including methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl and other higher carbon alkyl groups. Further examples include 2-methylpropyl, 2-methyl-4-ethylbutyl, 2,4-diethylpropyl, 3-propylbutyl, 2,8-dibutyldecyl, 6,6-dimethyloctyl, 6-propyl-6-butyloctyl, 2-methylbutyl, 2-methylpentyl, 3-methylpentyl, 2-ethylhexyl and other branched chain groups.

A number of substituent groups can be introduced into compounds of the invention in a protected (blocked) form and subsequently de-protected to form a final, desired compound. In general, protecting groups render chemical functionality inert to specific reaction conditions and can be appended to and removed from such functionality in a molecule without substantially damaging the remainder of the molecule. See, e.g., Greene and Wuts, Protective Groups in Organic Synthesis, 2d ed, John Wiley & Sons, New York, 1991. For example, amino groups can be protected as phthalimido groups or as 9-fluorenylmethoxycarbonyl (FMOC) groups and carboxyl groups can be protected as fluorenylmethyl groups. Representative hydroxyl protecting groups are described by Beaucage, et al., Tetrahedron 1992, 48, 2223. Preferred hydroxyl protecting groups are acid-labile, such as the trityl, monomethoxytrityl, dimethoxytrityl, and trimethoxytrityl groups.

Solid supports used for solid state synthesis of the oligonucleotides according to the invention include controlled pore glass (CPG), oxalyl-controlled pore glass (see, e.g., Alul, et al., Nucleic Acids Research 1991, 19, 1527), TentaGel Support an aminopolyethyleneglycol derivatized support (see, e.g., Wright, et al., Tetrahedron Letters 1993, 34, 3373) or Poros—a copolymer of polystyrene/divinylbenzene. Many other solid supports are commercially available and amenable to the present invention.

An activated solid support in the context of the present invention is a solid support that has been derivatized with a functional group or treated with a reactive moiety such that the resulting activated solid support is chemically active towards reaction with a monomeric unit for preparing the oligonucleotides of the invention. Standard methods and techniques used to increase the coupling efficiency of oligonucleotide synthesis include activation of 3′ and or 5′ functional groups. Some commonly activated groups are phosphate and phosphite which give the corresponding activated phosphate and activated phosphite (see e.g., Nucleic Acids in Chemistry and Biology; Blackburn, G. M., Gait M. J., Eds. Chemical Synthesis; IL: New York, 1990, Chapter 3, p. 98). Many others are known and can be used herein.

Monomeric sub-units of the invention are coupled using linking moieties. Linking moieties include phosphodiester, phosphotriester, hydrogen phosphonate, alkylphosphonate, alkylphosphonothioate, arylphosphonothioate, phosphorothioate, phosphorodithioate, phosphoramidate, ketone, sulfone, carbonate and thioamidate. Alkylphosphonothioate linkages are disclosed in WO 94/02499. Other such moieties can also be employed.

Peptide nucleic acids and locked nucleic acids may also be used as antisense binding materials. Both kinds are synthesized as complements to the appropriate DNA or RNA sequence as described above. The structures and processes for synthesis follow the descriptions of the following references, the disclosures of which are incorporated herein by reference: B. Hyrup, P. E. Nielsen, Peptide Nucleic Acids(PNA): Synthesis, Properties, and Potential Applications. Bioorg. Med. Chem. Lett. 4(1):5-23 (1996); J. Wengel et al., “LNA (Locked Nuceic Acid)”, Nucleosides, Nucleotides 18, 1365-70 (1999).

### Immobilized GPI

The enzyme GPI may be immobilized upon a support by covalently linking it through linking groups. Supports such as gels, dextrins, molecular beads, polymers and the like may be used. Linking to the carboxyl or amine terminus of GPI may be accomplished by esterification, amidation, Schiff base formation and the like. The length of the linking group may be adjusted to enable appropriate conformation of the enzyme and its binding to the AGPI Ab. Techniques for immobilizing proteins and enzymes such as GPI are well-known in the art. See for example, U.S. Pat. No. 5,234,820, the disclosure of which is incorporated herein by reference.

### GPI-Cvtotoxic Agent Conjugate

GPI may also be covalently bound to a cytotoxic agent to enable targeting of Bcells producing the AGPI Ab. Preferably, the epitope of GPI that binds to AGPI Ab is employed ether alone or as part of a polypeptide sequence that holds the epitope in a desired configuration. Preferably, the polypeptide is a segment of GPI.

The cytotoxic agents useful according to the invention include radioactive agents, cytotoxic cancer agents such as Ara-C (1-.beta.-D-arabinofuranosylcytosine), adriamycin, daunorubicin, vinblastine, etoposide, methotrexate, 5-fluorouracil, chlorambucil, cisplatin, and hydroxyurea, adriamycin, daunorubicin, vinblastine, etoposide, cell lysing agents and the like. Further cytotoxic agents include “chemotherapeutic agents” which are compounds having biological activity against one or more forms of cancer. Suitable chemotherapeutic agents include antineoplasts. Representative antineoplasts include adjuncts, androgen inhibitors, antibiotic derivatives, antiestrogen, antimetabolites, cytotoxic agents, hormones, immunomodulators, nitrogen mustard derivatives and steroids. Physicians' Desk Reference, 50th Edition, 1996.

Representative adjuncts include levamisole, gallium nitrate, granisetron, sargramostim strontium-89 chloride, filgrastim, pilocarpine, dexrazoxane, and ondansetron. Physicians' Desk Reference, 50th Edition, 1996.

Representative androgen inhibitors include flutamide and leuprolide acetate. Physicians' Desk Reference, 50th Edition, 1996.

Representative antibiotic derivatives include doxorubicin, bleomycin sulfate, daunorubicin, dactinomycin, and idarubicin.

Representative antiestrogens include tamoxifen citrate. Physicians' Desk Reference, 50th Edition, 1996.

Representative antimetabolites include fluorouracil, fludarabine phosphate, floxuridine, interferon alfa-2b recombinant, methotrexate sodium, plicamycin, mercaptopurine, and thioguanine. Physicians' Desk Reference, 50th Edition, 1996.

Representative cytotoxic agents include doxorubicin, carmustine [BCNU], lomustine [CCNU], cytarabine USP, cyclophosphamide, estramucine phosphate sodium, altretamine, hydroxyurea, ifosfamide, procarbazine, mitomycin, busulfan, cyclophosphamide, mitoxantrone, carboplati, cisplati, cisplatin, interferon alfa-2a recombinant, paclitaxel, teniposide, and streptozoci. Physicians' Desk Reference, 50th Edition, 1996.

Representative hormones include medroxyprogesterone acetate, estradiol, megestrol acetate, octreotide acetate, diethylstilbestrol diphosphate, testolactone, and goserelin acetate. Physicians' Desk Reference, 50th Edition, 1996.

Representative immunodilators include aldesleukin. Physicians' Desk Reference, 50th Edition, 1996.

Representative nitrogen mustard derivatives include melphalan, chlorambucil, mechlorethamine, and thiotepa. Physicians' Desk Reference, 50th Edition, 1996.

Representative steroids include betamethasone sodium phosphate and betamethasone acetate. Physicians' Desk Reference, 50th Edition, 1996.

Specifically, the chemotherapeutic agent is an antineoplastic agent.

Specifically, the antineoplastic agent is a cytotoxic agent.

Specifically, the cytotoxic agent is paclitaxel or doxorubicin.

Additional suitable chemotherapeutic agents include alkylating agents, antimitotic agents, plant alkaloids, biologicals, topoisomerase I inhibitors, topoisomerase II inhibitors, and synthetics. AntiCancer Agents by Mechanism, http://www.dtp.nci.nih.gov/docs/cancer/searches/standard_mechanism_list.html, Apr. 12, 1999; Approved Anti-Cancer Agents, http://www.ctep.info.nih.gov/handbook/HandBookText/fda_agen.htm, pages 1-7, Jun. 18, 1999; MCMP 611 Chemotherapeutic Drugs to Know, http//www.vet.purdue.edu/depts/bms/courses/mcmp611/chrx/drg2no61.html, Jun. 24, 1999; and Chemotherapy, http://www.vetmed.lsu.edu/oncology/Chemotherapy.htm, Apr. 12, 1999.

Representative alkylating agents include asaley, AZQ, BCNU, busulfan, bisulphan, carboxyphthalatoplatinum, CBDCA, CCNU, CHIP, chlorambucil, chlorozotocin, cis -platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864. AntiCancer Agents by Mechanism, http ://dtp.nci.nih. gov/docs/cancer/searches/standard_mechanism_list.html, Apr. 12, 1999.

Representative antimitotic agents include allocolchicine, Halichondrin B, colchicine, colchicine derivatives, dolastatin 10, maytansine, rhizoxin, paclitaxel derivatives, paclitaxel, thiocolchicine, trityl cysteine, vinblastine sulfate, and vincristine sulfate. AntiCancer Agents bv Mechanism, http://dtp.nci.nih.gov/docs/cancer/searches/standard_mechanism_list.html, Apr. 12, 1999.

Representative plant alkaloids include actinomycin D, bleomycin, L-asparaginase, idarubicin, vinblastine sulfate, vincristine sulfate, mitramycin, mitomycin, daunorubicin, VP-1 6-213, VM-26, navelbine and taxotere. Approved Anti-Cancer Agents, http://ctep.info.nih.gov/handbook/HandBookText/fda_agent.htm, Jun. 18, 1999.

Representative biologicals include alpha interferon, BCG, G-CSF, GM-CSF, and interleukin-2. Approved Anti-Cancer Agents, http://ctep.info.nih.gov/handbook/HandBookText/fda_agent.htm, Jun. 18, 1999.

Representative topoisomerase I inhibitors include camptothecin, camptothecin derivatives, and morpholinodoxorubicin. AntiCancer Agents by Mechanism, http://dtp.nci.nih.gov/docs/cancer/searches/standard_mechanism_list.html, Apr. 12, 1999.

Representative topoisomerase II inhibitors include mitoxantron, amonafide, m-AMSA, anthrapyrazole derivatives, pyrazoloacridine, bisantrene HCL, daunorubicin, deoxydoxorubicin, menogaril, N, N-dibenzyl daunomycin, oxanthrazole, rubidazone, VM-26 and VP-16. AntiCancer Agents by Mechanism, http :/dtp.nci.nih.gov/docs/cancer/searches/standard_mechanism_list.html, Apr. 12, 1999.

Representative synthetics include hydroxyurea, procarbazine, o,p=-DDD, dacarbazine, CCNU, BCNU, cis-diamminedichloroplatimun, mitoxantrone, CBDCA, levamisole, hexamethylmelamine, all-trans retinoic acid, gliadel and porfimer sodium. Approved Anti-Cancer Agents, http://ctep.info.nih.gov/handbook/HandBookText/fda_agen.htm, Jun. 18, 1999.

The conjugation through covalent binding may be produced according to the methods disclosed in U.S. Pat. No. 5,234,820 and E. T. Koh et al., Biotechniques, 7, 596 et seq. (1989).

### Diagnosis and Treatment With The Immunopolypeptides

The immunopolypeptides, anti-idiotypic antibodies to AGPI Ab, the second immunopolypeptides, the antisense oligonucleotides, the GPI alone or in combination with adjuvants (hereinafter the therapeutic agents of the invention) may generally be formulated with a pharmaceutically acceptable carrier and may be administered by any desired route. More particularly, the therapeutic agents of the invention may be formulated with a buffered aqueous, oil or organic medium containing optional stabilizing agents and adjuvants for stimulation of immune binding. A preferred formulation involves lyophilized therapeutic agent and separate pharmaceutical carrier. Immediately prior to administration, the formulation is constituted by combining the lyophilized therapeutic agent and pharmaceutical carrier. Administration by a parenteral or oral regimen will deliver the immunopolypeptide to the desired site of action. The dosage and route of administration will generally follow the judgment of the patient's attending physician. In particular, intravenous, intraperitoneal, intramuscular, subcutaneous, rectal or vaginal administration may be used.

Pharmaceutical formulations of the therapeutic agents of the invention can prepared as liquids, gels and suspensions. The formulations are preferably suitable for injection, insertion or inhalation. Injection may be accomplished by needle, cannula catheter and the like. Insertion may be accomplished by lavage, trochar, spiking, surgical placement and the like. Inhalation may be accomplished by aerosol, spray or mist formulation. The immunopolypeptide of the invention may also be administered topically such as to the epidermis, the buccal cavity and instillation into the ear, eye and nose.

The carrier for the pharmaceutical formulations includes any pharmaceutically acceptable agent suitable for delivery by any one of the foregoing routes and techniques of administration. Diluants, stabilizers, buffers, adjuvants, surfactants, fungicides, bactericides, and the like may also optionally be included. Such additives will be pharmaceutically acceptable and compatible with the therapeutic agent of the invention. Carriers include aqueous media, buffers such as bicarbonate, phosphate and the like; ringers solution, Ficol solution, BSA solution, EDTA solution, glycerols, oils of natural origin such as almond, corn, arachnis, caster or olive oil; wool fat or its derivatives, propylene glycol, ethylene glycol, ethanol, macrogols, sorbitan esters, polyoxyethylene derivatives, natural gums, and the like.

Therapeutic techniques useful for treatment of rheumatoid arthritis rely upon interception or prevention of the immunogenic activity of AGPI Ab. The immunopolypeptides of the invention may be administered to an afflicted patient to bind with the epitopal sites of GPI thus preventing the immune reaction with AGPI Ab. The anti-idiotypic antibodies and second immunopolypeptides of the invention may be administered to an afflicted patient to bind the AGPI Ab thus preventing the immune reaction with GPI. The antisense oligonucleotides may be administered to an afflicted patient to prevent or ameliorate expression of AGPI Ab thus preventing the antibody GPI immune reaction. The antisense oligonucleotides to the GPI nucleotide sequence may be administered to an afflicted patient by a titration dosing regimen designed to identify the level of antisense oligonucleotide needed to bring the GPI tissue level to normal. Typically that tissue level will be measured by a diagnostic technique conducted upon tissue from afflicted sites such as joints and/or connective tissue. The immobilized GPI may be used to filter excorporeally an afflicted patient's blood and remove AGPI Ab. The conjugate, complex and the like formed by binding GPI or an epitopal site thereof to a cytotoxic agent may be administered to an afflicted patient to eliminate those B cells producing AGPI Ab. The formulation of GPI for desensitization may be administered to an afflicted patient to cause the patient's immune system to self adjust to the presence of GPI.

The amount of therapeutic agent useful to establish appropriate treatment of rheumatoid arthritis according to the therapeutic technique associated with the agent can be determined by diagnostic and therapeutic techniques well known to those of ordinary skill in the art. For example, for the immunopolypeptides, chimeric monoclonal antibodies, second immunopolypeptides and antisense oligonucleotides, the dosage may be determined by titrating a sample of the patient's blood sera with the selected therapeutic agent to determine the end point beyond which no further immunocomplex is formed. For desensitization treatment, the dosage may be started at a low level such as 1 microgram per kg of body weight and increased until an allergic reaction is obtained. Such titrations may be accomplished by the diagnostic techniques discussed below. Available dosages include administration of from about 1 to about 1 million effective units of antibody per day, wherein a unit is that amount of therapeutic agent, which will provide at least 1 microgram of antigen-immunopolypeptide complex. Preferably, from about 100 to about 100,000 units of antibody per day can be administered. Alternatively, the immunopolypeptide of the invention may be administered in a range of about 0.05 to about 100, preferably 0.5 to about 50 mg per kg of patient body weight per day.

The therapeutic agents of the invention may be present in the pharmaceutical formulation at concentrations ranging from about 1 percent to about 50 percent, preferably about 1 percent to about 20 percent, more preferably about 2 percent to about 10 percent by weight relative to the total weight of the formulation.

For extracorporeal filtering, the immobilized GPI may be contained in a sterile filtration system attached to a device for removal and re-introduction of a patient's blood. The patient is connected to the system through invasion of a convenient vein and the blood filtered until no further GPI reaction is observed. The GPI reaction may be observed through the diagnostic technique described below.

Diagnostic and screening techniques useful for identification of patients afflicted with rheumatoid arthritis or having a propensity for development of rheumatoid arthritis include any that identify antibody-antigen binding. A GPI sample can be combined with an appropriate sample from the patient to produce a complex. The complex in turn can be detected with a marker reagent for binding with such a complex. Typical marker reagents include antibodies selective for the complex, antibodies selective for certain epitopes of the AGPI Ab or a label attached to the GPI itself. In particular, radioimmunoassay (RIA), radioallergosorbent test (RAST), radioimmunosorbent test (RIST), immunradiometric assay (IRMA) Farr assay, fluorescence immunoassay (FIA), sandwich assay, enzyme linked immunosorbent assay (ELISA) assay, northern or southern blot analysis, and color activation assay may be used following protocols well known for these assays. See for example Immunology, An Illustrated Outline by David Male, C. V. Mosby Company, St Louis, Mo., 1986 and the Cold Spring Harbor Laboratory Manuals cited above. Labels including radioactive labels, chemical labels, fluorescent labels, luciferase and the like may also be directly attached to GPI according to the techniques described in U.S. Pat. No. (BN patent cite), the disclosure of which is incorporated herein by reference.

Alternatively and perhaps more practically, to ensure long antibody half-lives, an intact antibody with inert effector function, such as an IgG2 antibody or an IgGI antibody with altered effector function may be used ³³. It may be therapeutically beneficial to administer the anti-GPI antibody in conjunction with available and successful anti-TNF blocking antibodies³⁴.

### Definitions

Terms used throughout this application are to be construed with ordinary and typical meaning to those of ordinary skill in the art. The following terms are to be given the particular definitions given below.

The term “immunopolypeptide” refers to a chain of two (2) or more amino acids which are linked together with peptide or amide bonds, regardless of post-translational modification (e.g., glycosylation or phosphorylation). Antibodies are specifically intended to be within the scope of this definition. The immunopolypeptides of this invention may include more than one subunit, where each subunit is encoded by a separate DNA sequence.

The phrases “significant homology”, “substantially homogeneous”, significant homolog”, “significantly homogeneous” and “substantial identity” with respect to an antibody or immunopolypeptide sequence mean an antibody or immunopolypeptide sequence exhibiting at least 80%, preferably 90%, more preferably 95% and most preferably 99% sequence identity to the reference antibody or immunopolypeptide sequence. The term with respect to a nucleic acid sequence means a sequence of nucleotides exhibiting at least about 80%, preferably 90%, more preferably 95% and most preferably 99% sequence identity to the reference nucleic acid sequence. For immunopolypeptides, the length of the comparison sequences will generally be at least 25 amino acids. For nucleic acids, the length will generally be at least 75 nucleotides.

The term “identity” or “homology” means the percentage of amino acid residues in the candidate sequence that are identical with the residue of a corresponding sequence to which it is compared, after aligning the sequences and introducing gaps, if necessary to achieve the maximum percent identity for the entire sequence, and not considering any conservative substitutions as part of the sequence identity. Neither N- or C- terminal extensions nor insertions shall be construed as reducing identity or homology. Methods and computer programs for the alignment are well known in the art. Sequence identity may be measured using sequence analysis software (e.g., Sequence Analysis Software Package, Genetics Computer Group, University of Wisconsin Biotechnology Center, 1710 University Ave., Madison, Wis. 53705). This software matches similar sequences by assigning degrees of homology to various substitutions, deletions, and other modifications.

The term “antibody” is used in the broadest sense, and specifically covers monoclonal antibodies (including full length monoclonal antibodies), polyclonal antibodies, multispecific antibodies (e.g., bispecific antibodies), and antibody fragments (e.g., Fab, F(ab')₂, Fd and Fv) so long as they exhibit the desired biological activity.

Native antibodies are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable region (V_{H}) followed by a number of constant regions. Each light chain has a variable region at one end (V_{L}) and a constant region at its other end. The constant region of the light chain is aligned with the first constant region of the heavy chain, and the light chain variable region is aligned with the variable region of the heavy chain. The variable region of either chain has a triplet of hypervariable or complementarity determining regions (CDR's) spaced within a framework sequence as explained below. The framework and constant regions of the antibody have highly conserved amino acid sequences such that a species consensus sequence may typically be available for the framework and constant regions. Particular amino acid residues are believed to form an interface between the light and heavy chain variable regions (Chothia et al., J. Mol. Biol. 186, 651-63, 1985); Novotny and Haber, Proc. Natl. Acad. Sci. USA 82 4592-4596 (1985).

The term “variable” in the context of variable region of antibodies, refers to the fact that certain portions of the variable regions differ extensively in sequence among antibodies and are used in the binding and specificity of each particular antibody for its particular antigen. The variability is concentrated in three segments (a triplet) called complementarity determining regions (CDRs) also known as hypervariable regions both in the light chain and the heavy chain variable regions. There are at least two techniques for determining CDRs: (1) an approach based on cross-species sequence variability (i.e., Kabat et al., Sequences of Proteins of Immunological Interest (National Institute of Health, Bethesda, Md. 1987); and (2) an approach based on crystallographic studies of antigen-antibody complexes (Chothia, C. et al. (1989), Nature 342: 877).

The more highly conserved portions of variable regions are called the framework (FR). The variable domains of native heavy and light chains each comprise three FR regions, largely adopting a β-Sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies (see Kabat et al.) The constant regions and Fc are not involved directly in binding an antibody to an antigen, but exhibit various effector function, such as participation of the antibody in antibody-dependent cellular toxicity.

A “species-dependent antibody,” e.g., a mammalian anti-human IgE antibody, is an antibody which has a stronger binding affinity for an antigen from a first mammalian species than it has for a homologue of that antigen from a second mammalian species. Normally, the species-dependent antibody “bind specifically” to a human antigen (i.e., has a binding affinity (Kd) value of no more than about 1×10⁻⁷ M, preferably no more than about 1×10⁻⁸ and most preferably no more than about 1×10⁻⁹ M) but has a binding affinity for a homologue of the antigen from a second non-human mammalian species which is at least about 50 fold, or at least about 500 fold, or at least about 1000 fold, weaker than its binding affinity for the human antigen. The species-dependent antibody can be of any of the various types of antibodies as defined above, but preferably is a humanized or human antibody.

The term “antibody variation” refers to an amino acid sequence variant of an antibody wherein one or more of the amino acid residues have been modified. Such a variation necessarily has less than 100% sequence identity or similarity with the original amino acid sequence. Preferably it has at least 75% amino acid sequence identity or similarity with the original amino acid sequence of either the heavy or light chain variable domain of the antibody of which it is a variation, more preferably at least 80%, more preferably at least 85%, more preferably at least 90%, and most preferably at least 95%.

The term “antibody fragment” refers to a portion of a full-length antibody, generally the antigen binding or variable region. Examples of antibody fragments include Fab, Fab', F(ab')₂, Fd and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab')₂ fragment that has two antigen binding fragments which are capable of crosslinking antigen, and a residual other fragment (which is termed pFc'). Additional fragments can include diabodies, linear antibodies, single-chain antibody molecules, and multispecific antibodies formed from antibody fragments. As used herein, “functional fragment” with respect to antibodies, refers to Fv, F(ab) and F(ab')₂ and Fd fragments.

An “Fv” fragment is the minimum antibody fragment which contains a complete antigen recognition and binding site. This region consists of a dimer of one heavy and one light chain variable domain in a tight, non-covalent association (V_{H}-V_{L} dimer). It is in this configuration that the three CDRs of each variable region interact to define an antigen binding site on the surface of the V_{H}-V_{L} dimer. Collectively, the six CDRs confer antigen binding specificity to the antibody. However, even a single variable region (or half of an Fv comprising only three CDRs specific for an antigen) has the ability to recognize and bind antigen, although at a lower affinity than the entire binding site.

The Fab fragment (also designated as F(ab)) also contains the constant region of the light chain and the first constant region (CH1) of the heavy chain. Fab' fragments differ from Fab fragments by the addition of a few residues at the carboxyl terminus of the heavy chain CH1 domain including one or more cysteines from the antibody hinge region. Fab'-SH is the designation herein for Fab' in which the cysteine residue(s) of the constant regions have a free thiol group. F(ab') fragments are produced by cleavage of the disulfide bond at the hinge cysteines of the F(ab')₂ pepsin digestion product. Additional chemical couplings of antibody fragments are known to those of ordinary skill in the art.

The light chains of antibodies (immunoglobulin) from any vertebrate species can be assigned to one of two clearly distinct types, called kappa (κ) and lambda (λ), based on the amino sequences of their constant domain.

Depending on the amino acid sequences of the constant domain of their heavy chains, “immunoglobulins” can be assigned to different classes. There are at least five (5) major classes of immunoglobulins: IgA, IgD, IgE, IgG and IgM, and several of these may be further divided into subclasses (isotypes), e.g. IgG-1, lgG-2, IgG-3 and IgG4; IgA-1 and IgA-2. The heavy chains constant domains that correspond to the different classes of immunoglobulins are called .alpha., .delta., .epsilon., and gamma. and .mu., respectively. The subunit structures and three-dimensional configurations of different classes of immunoglobulins are well known. The preferred immunoglobulin for use with the present invention is immunoglobulin IgG.

The term “monoclonal antibody” as used herein as a subclass of the immunopolypeptide of the invention refers to an antibody obtained from a population of substantially homogeneous antibodies, i.e., the individual antibodies composed of the population are identical except for possible naturally occurring mutations that may be present in minor amounts. Monoclonal antibodies are highly specific, being directed against a single antigenic site. Furthermore, in contrast to conventional (polyclonal) antibody preparations, which typically include different antibodies, directed against different determinants (epitopes), each monoclonal antibody is directed against a single determinant on the antigen. In additional to their specificity, the monoclonal antibodies are advantageous in that they are synthesized by a hybridoma or phage infected bacterial culture, uncontaminated by other immunoglobulins. The modifier “monoclonal” indicates the character of the antibody indicates the character of the antibody as being obtained from a substantially homogeneous population of antibodies, and is not to be construed as requiring production of the antibody by any particular method. For example, the monoclonal antibodies may be made by the hybridoma method first described by Kohler and Milstein, Nature 256, 495 (1975), or may be made by recombinant methods, e.g., as described in U.S. Pat. No. 4,816,567. The monoclonal antibodies for use with the present invention may also be isolated from phage antibody libraries using the techniques described in Clackson et al. Nature 352: 624-628 (1991), as well as in Marks et al., J. Mol. Biol. 222: 581-597 (1991).

Chimeric antibodies are antibodies whose light and heavy chain genes have been constructed, typically by genetic engineering, from immunoglobulin variable and constant region genes belonging to different species. For example, the variable segments of the genes from a mouse monoclonal antibody may be joined to human constant segments, such as gamma 1 and gamma 3. A typical therapeutic chimeric antibody is thus a hybrid protein composed of the variable or antigen-binding domain from a mouse antibody and the constant or effector domain from a human although other mammalian species may be used.

As used herein, the term “humanized” immunoglobulin or antibody refers to an immunoglobulin or antibody composed of a human framework region and one or more CDR's from a non-human (usually a mouse or rat) immunoglobulin. The non-human immunoglobulin providing the CDR's is called the “donor” and the human immunoglobulin providing the framework is called the “acceptor”. Constant regions need not be present, but if they are, they must be substantially identical to human immunoglobulin constant regions, i.e., at least about 85-90%, preferably about 95% or more identical. Hence, all parts of a humanized immunoglobulin, except possibly the CDR's, are substantially identical to corresponding parts of natural human immunoglobulin sequences. A “humanized antibody” is an antibody comprising a humanized light chain and a humanized heavy chain immunoglobulin. For example, a humanized antibody would not encompass a typical chimeric antibody as defined above, e.g., because the entire variable region of a chimeric antibody is non-human. One says that the donor antibody has been “humanized”, by the process of “humanization”, because the resultant humanized antibody is expected to bind to the same antigen as the donor antibody that provides the CDR's.

It is understood that the humanized antibodies designed according to the present invention may have additional conservative amino acid substitutions which have substantially no effect on antigen binding or other immunoglobulin functions.

By conservative substitutions is intended combinations such as gly, ala; val, ile, leu; asp, glu; asn, gin; ser, thr; lys, arg; and phe, tyr.

The immunopolypeptide subclasses including monoclonal antibodies, fragments and single chains thereof include “chimeric” forms in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567); Morrison et al. Proc. Natl. Acad. Sci. 81, 6851-6855 (1984).

The immunopolypeptide subclasses also include fully human forms in which the entire sequence is derived from human immunoglobulins (recipient antibody) including the complementary determining region (CDR) of the immunopolypeptide In some instances, Fv framework residues of the human immunoglobulin are replaced by corresponding non-human residues. Furthermore, an immunopolypeptide include residues, which are found neither in a human immunoglobulin nor in a non-human mammalian sequence.

“Single-chain Fv” or “scFv” antibody fragments include the V_{H} and V_{L} regions of an antibody, wherein these regions are present in a single immunopolypeptide chain. Generally, the Fv immunopolypeptide further includes an immunopolypeptide linker between the V_{H} and V_{L} regions which enables the scFv to form the desired structure for antigen binding. For a review of scFv see Pluckthun in The Pharmacology of Monoclonal Antibodies, vol. 113, Rosenburg and Moore eds. Springer-Verlag, New York, pp. 269-315 (1994).

The term “diabodies” refers to a small antibody fragments with two antigenbinding sites, which fragments comprise a heavy chain variable region (V_{H}) connected to a light chain variable domain (V_{L}) in the same immunopolypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161, and Holliger et al., Proc. Natl. Acad. Sci. USA 90: 6444-6448 (1993).

The term “amino acid” and “amino acids” refer to all naturally occurring L-α-amino acids.

The term “Variants” refers to substitutional, insertional and/or deletional variants. “Substitutional” variants are those that have at least one amino acid residue in a native sequence removed and a different amino acid inserted in its place at the same position. The substitutions may be single, where only one amino acid in the molecule has been substituted, or they may be multiple, where two or more amino acids have been substituted in the same molecule. “Insertional” variants are those with one or more amino acids inserted immediately adjacent to an amino acid at a particular position in a native sequence. Immediately adjacent to an amino acid means connected to either the .alpha.-carboxyl or .alpha.-amino functional group of the amino acid. “Deletional” variants are those with one or more amino acids in the native amino acid sequence removed. Ordinarily, deletional variants will have one or two amino acids deleted in a particular region of the molecule.

The terms “cell”, “cell line” and “cell culture” are used interchangeably, and all such designations include progeny. It is also understood that all progeny may not be precisely identical in DNA content, due to deliberate or inadvertent mutations. Mutant progeny that have the same function or biological property, as screened for in the originally transformed cell, are included.

The “host cells” used in the present invention generally are prokaryotic or eukaryotic hosts.

“Transformation” means introducing DNA into an organism so that the DNA is replicable, either as an extrachromosomal element or by chromosomal integration.

“Transfection” refers to the taking up of an expression vector by a host cell whether or not any coding sequences are in fact expressed.

The terms “transfected host cell” and “transformed” refer to the introduction of DNA into a cell. The cell is termed “host cell” and it may be either prokaryotic or eukaryotic. Typical prokaryotic host cells include various strains of E. coli. Typical eukaryotic host cells are mammalian, such as Chinese hamster ovary or cells of human origin. The introduced DNA sequence may be from the same species as the host cell of a different species from the host cell, or it may be a hybrid DNA sequence, containing some foreign and some homologous DNA.

The terms “replicable expression vector” and “expression vector” refer to a piece of DNA, usually double-stranded, which may have inserted into it a piece of foreign DNA. Foreign DNA is defined as heterologous DNA, which is DNA not naturally found in the host cell. The vector is used to transport the foreign or heterologous DNA into a suitable host cell. Once in the host cell, the vector can replicate independently of the host chromosomal DNA and several copies of the vector and its inserted (foreign) DNA may be generated.

The term “vector” means a DNA construct containing a DNA sequence, which is operably linked to a suitable control sequence capable of effecting the expression of the DNA in a suitable host. Such control sequences include a promoter to effect transcription, an optional operator sequence to control such transcription, a sequence encoding suitable mRNA ribosome binding sites, and sequences that control the termination of transcription and translation. The vector may be a plasmid, a phage particle, or simply a potential genomic insert. Once transformed into a suitable host, the vector may replicate and function independently of the host genome, or may in some instances, integrate into the genome itself. In the present application, “phage” and “vector” are sometimes used interchangeably, as the phage is the form of vector used in the present invention. However, the term vector is intended to include such other form of vectors which serve equivalent function as and which are, or become, known in the art. Typical expression vectors for bacterial expression and mammalian cell culture expression, for example, are based on pRK5 (EP 307,247), pSV16B (WO 91/08291) and pVL1392 (Pharmingen).

The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

An “isolated” nucleotide sequence is a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguishable from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

A nucleotide sequence is “operably linked” when it is placed into a functional relationship with another nucleic acid sequence. This can be a gene and a regulatory sequence(s) which are connected in such a way as to permit gene expression when the appropriate molecules (e.g., transcriptional activator proteins) are bound to the regulatory sequences(s). For example, DNA for a presequence or secretory leader is operably linked to DNA for an immunopolypeptide if it is expressed as a preprotein that participates in the secretion of the immunopolypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Treatment” refers to both therapeutic treatment and prophylactic or preventative measures. Those in need of treatment include those already with the disorder as well as those in which the disorder is to be prevented.

A “disorder” is any condition that would benefit from treatment with the immunopolypeptide. This includes chronic and acute disorders or diseases including those pathological conditions which predispose the mammal to the disorder in question.

The word “label” when used herein refers to a detectable compound or composition which is conjugated directly or indirectly to the antibody. The label may itself be detectable (e.g., radioisotope labels or fluorescent labels) or, in the case of an enzymatic label, may catalyze chemical alteration of a substrate compound or composition which is detectable.

As used herein, “solid phase” means a non-aqueous matrix to which the antibody of the present invention can adhere. Example of solid phases encompassed herein include those formed partially or entirely of glass (e.g. controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g. an affinity chromatography column). This term also includes a discontinuous solid phase of discrete particles, such as those described in U.S. Pat. No. 4,275,149.

As used herein, “affinity maturation using phage display” (AMPD) refers to a process described in Lowman et al., Biochemistry 30(45): 10832-10838 (1991), see also Hawkins et al., J. Mol Biol. 226, 889-896 (1992). While not strictly limited to the following description, this process can be described briefly as: several hypervariable region sites (e.g. 6-7 sites) are mutated to generate all possible amino acid substitutions at each site. The antibody mutants thus generated are displayed in a monovalent fashion from filamentous phage particles as fusions to the gene III product of M13 packaged within each particle. The phage expressing the various mutants can be cycled through rounds of binding selection, followed by isolation and sequencing of those mutants which display specific immunobinding, preferably high affinity binding. The method is also described in WO 92/09690, published Jun. 11, 1992. A modified procedure involving pooled affinity display is described in Cunningham, B. C. et al., EMBO J. 13(11), 2508-2515 (1994).

As used herein, the term “phage library” refers to the phage library used in the affinity maturation process described above and in Hawkins et al., J. Mol Biol. 226: 889-896 (1992), and in Lowman et al., Biochemistry 30(45): 10832-10838 (1991). Each library includes a variable region (e.g. 6-7 sites) for which all possible amino acid substitutions are generated. The antibody mutants thus generated are displayed in a monovalent fashion from filamentous phage particles as fusions to the gene III product of M13 packaged within each particle and expressed on the exterior of the phage.

As used herein, “high affinity” means an affinity constant (Kd) of at least 10⁻⁵ M and preferably at least 10⁻⁷ M, and especially preferably at least 10⁻¹⁰ M under physiological conditions.

The following discussion and examples further illustrate the invention. They are not meant to be general limitations of the invention, however, as the invention is fully set forth in the foregoing description.

### EXPERIMENTAL DISCUSSION AND EXAMPLES

### Discussion of the Investigation of GPI and its Autoantibody Reaction in Rheumatoid Arthritis

Mathis and colleagues showed, in a TCR transgenic arthritis mouse model ⁶, that GPI can serve as an agent interacting with both B and T cells.⁵,¹⁴. A discovery associated with the present invention is the revelation that GPI is an important autoantigen in human RA. Heretofore, no connection has been established between the mouse model and human rheumatoid arthritis or the GPI is an agent involved in human autoimmune disease. The invention establishes this connection and demonstrates that high titered anti-GPI IgG antibodies are found both in serum and synovial fluid of most RA patients. Significantly, the invention provides agents for intervening in the progress of autoimmune disease. In the following discussion, the discovery associated with the present invention is presented.

To characterize these antibodies, an IgG phage display library was generated from the bone marrow of a patient with high serum titer for GPI. Bone marrow has been found to be a major repository for plasma cells that produce antibodies, broadly reflecting the repertoire found in serum 15. Synovial tissue could also have been used. However since the number of plasma cells in this tissue is considerably lower, generation of a library of sufficient size would be more difficult. Specific anti-GPI antibodies selected from the library were shown to be of high affinity and to show evidence of extensive somatic mutation with high R/S ratios in the CDR regions, suggesting they are produced as a result of a matured immune response to GPI. Such a maturation process requires CD4⁺ help, so that, although we have not characterized these cells in this study, CD4⁺ cells recognizing GPI peptides in a MHC class II-dependent fashion are likely to exist in RA patients.

To determine how an autoimmune response against the ubiquitously expressed protein GPI could precipitate disease specifically within the joints, we examined the distribution of GPI in synovial tissue from patients with active RA synovitis compared to patients with non-inflammatory osteoarthritis or traumatic joint injuries. RA synovium is macroscopically characterized by increased vascularity, with pronounced blood cell vessel markings, granularity and villous formation (hypertrophy). Recently, angiogenesis in RA synovium has received a lot of attention, since it has been recognized that the endothelial cells lining the blood vessels play an important role in a variety of inflammatory and immunologic processes, including presentation of antigen to other immune cells, cytokine production, cellular adhesion, angiogenesis and recruitment of different inflammatory cells cell types to the synovium ¹⁶. Morphology and protein expression in the blood vessels of the synovium differ in RA and arthritis caused by other etiologies^{8;16}. In addition, the arterioles and venules serve different additional functions in the RA synovium and express different markers. Interest has been focused on the venules since some of the earliest changes in RA are endothelial swelling and transformation of small venules to high endothelial venules (HEV), i.e. those having high endothelial cells. The transformed venules provide a means for leukocytes to exit the blood stream into the synovium¹⁷. Our immunohistochemical studies revealed that intense staining for GPI in the inflamed RA synovium corresponded to the endothelial cell luminal surface, whereas no staining was found in osteoarthritis or normal synovium. Interestingly, the endothelial cell surface staining was restricted to the arterioles and some capillaries, but was not found in the small venules of the RA tissue. These results suggest that the arterioles may also play an important role in RA pathogenesis.

Intense staining for GPI was also observed on the surface of the synovial lining. The staining was not uniform along the surface of the synovial lining, but patchy and corresponded to the cytoplasm of some cells within the surface layer of the synovium. Particularly strong staining corresponded to the hyperplastic synovial villous. Interestingly, the staining pattern described here for GPI within the RA synovium resembles that observed for vascular permeability factor (VPF). VPF is a potent microvascular permeability enhancing cytokine that also exhibits selective endothelial cell mitogen activity and promotes angiogenesis in vivo ¹⁸. Within inflamed RA synovium but not in osteoarthritis synovium, VPF has been found in fibroblast-like type B synoviocytes and corresponding to the microvascular endothelium surface, where it is bound to its upregulated receptors (KDR and flt-1)¹⁹. Although many questions remain unanswered, a scenario similar to that found for VPF may occur for GPI. Selective cells in active synovium are stimulated to synthesize and secrete high levels of GPI into the synovial fluid. From the synovial fluid, GPI spills over into the circulation, which may explain the significantly higher GPI concentrations observed in RA synovial fluid compared to RA serum. In the arterioles and capillaries, GPI may bind to an upregulated receptor, one of which has been cloned from fibrosarcoma cells ²⁰. Binding of soluble GPI to a cell surface receptor is the probable reason for the observed GPI endothelial surface staining, since GPI is not a surface protein. Interestingly, GPI has been shown to stimulate cell migration and to exhibit cytokine and growth factor functions ²¹, suggesting that it may also be involved in recruitment of inflammatory cells to the synovium.

In addition to the cellular staining at the RA synovium surface lining, patchy noncellular staining was also found on the top of the surface lining, probably corresponding to precipitated immune complexes. GPI-containing immune complexes were also found in the synovial fluid, probably initiated by the binding of anti-GPI antibodies to the increased concentration of soluble GPI.

Thus, the results indicate that GPI may be presented to the immune system at both the endothelial surface and as soluble protein at high concentration particularly in the synovial fluid of the inflamed RA joint, but also in the circulation of RA patients. The two different presentations of GPI may lead to two different forms of immune attack. While the circulating affinity-matured anti-GPI IgG antibodies can directly bind to GPI exposed on the endothelial surface, the antibodies in the synovial fluid can form immune complexes with soluble GPI which is then precipitated on synovial lining with subsequent activation of the complement cascade. Whether either or both of these mechanisms are important for RA joint pathology requires further investigation.

Another critical issue is why tolerance to GPI is broken. The mechanism responsible for the onset of autoimmunity is difficult to trace, since it may occur long before the onset of clinical signs of the disease and may involve several factors other than the state of immune cells, such as genetic background and environmental factors. GPI, although an important intracellular antigen, also has extracellular functions, and GPI is found at constant low, but significant, levels (0.04-0.15 U/ml) in the serum of healthy individuals ¹³. Thus, T and B cells should be continuously exposed to this antigen and autoreactive anti-GPI cells should either be deleted in the thymus or be anergic. Loss of tolerance may occur after pathogenic infection as a result of molecular mimicry, epitope spreading or bystander activation 3;22;23. In addition, pathogens may express GPI with partial homology to human GPI, leading to immune recognition of non-self GPI and possible cross-reactivity with self GPI. Tolerance breakdown may also occur by activation of ignorant T or B cells upon viral infection, protein immunization or stimulation with polyclonal activators²⁴⁻²⁶. A breakdown of B cell tolerance has also been observed in anergic cells after removal of self-antigen ²⁷ or when T cell help was provided at the time of initial self-antigen encounter^{28;29}.

The identification of GPI as the target of the linked T/B cell response in K/BxN TCR transgenic mice and in humans with RA was somewhat of a surprise. Mathis and colleagues interpreted the K/BxN mouse model data as support for the contention that an organ-specific disease could result from systemic self-reactivity since GPI is ubiquitously expressed. Whilst GPI is ubiquitously expressed intracellularly, extracellular and membrane-bound GPI appear to be more localized. Increased levels of GPI are found locally in synovial fluid of RA patients and at the endothelial surfaces of the RA synovium and this may have important implications for the specificity of the autoimmune attack.

Interestingly, patients with high GPI levels also exhibit high anti-GPI IgG antibody levels and the two may be causally linked. However, the increased GPI levels could also be a consequence of increased tissue damage in the patients with high levels of arthritogenic anti-GPI antibodies. On the other hand, it is not likely that the increased levels of soluble GPI alone are responsible for the anti-GPI response. Increased levels of GPI have been observed in various types of cancer such as esophageal, gastric and lung carcinomas and acute myeloid leukemia (AML), but not in colon cancer, and GPI has been proposed as a diagnostic marker for these cancers ^{10;30-32}. In AML and lung cancer, the reported serum levels of GPI were in the same range as the RA patients in the present studies, but no consistent arthritis symptoms have been reported in these cancer patients.

As expected, anti-GPI antibodies are not found in all RA patients. Rheumatoid arthritis, although defined by a number of specific criteria set by the American College of Rheumatology, is a very heterogeneous disease and several different pathways are likely to result in similar clinical features. This also seems evident from RA studies in animal models wherein similar manifestations can be observed following induction of arthritis with distinct eliciting agents.

### Serum of RA patients contain high levels of anti-GPI IgG antibodies

To investigate whether patients with different autoimmune diseases and normal healthy individuals had serum antibodies against GPI, panels of sera from different groups of patients were titered for binding to purified GPI in ELISA. Anti-GPI IgG antibodies were found in the majority of the RA patients as 44 of 69 sera (64%) were considered positive whereas only 3 of 107 sera (3%) of the healthy normal donors were considered weakly positive (FIG. 1A). No correlation between positivity for GPI and age or gender of healthy normal donors was observed. Positivity was defined as an OD 405 value more than two standard deviation above the mean of the control normal donor values (>1.33) at a serum dilution of 1:50. Seventeen sera from patients with Lyme's arthritis and 22 from patients with Sjögren's syndrome without RA were also tested for anti-GPI antibodies, exhibiting only one clear positive and two marginal positives in each group. The mean level of anti-GPI antibodies in the RA patients group (OD405 nm: 1.83+/−0.84) was significantly higher than that of the normal healthy donor group (0.59+/−0.37)(p<0.0001), the Lyme's arthritis group (0.73+/−0.35)(p<0.0001), or the Sjögren's syndrome group (0.82+/−0.56)(p<0.0001). A subgroup of patients with RA had spontaneous sustained neutropenia of <2.0×10⁹/L and were classified as having Felty's syndrome. However, no difference in frequency of anti-GPI IgG antibodies was observed between the patients with or without Felty's syndrome. The level of anti-GPI IgG antibodies was very high in some of the RA sera exhibiting positive GPI reactivity at titers of 1:6,400.

An AP-conjugated F(ab')₂ fragment of a goat anti-human IgG Fc specific antibody was used as secondary antibody to avoid problems of rheumatoid factor binding in the serum samples. The serum samples were also tested for the presence of rheumatoid factor, demonstrating that 73% of the RA sera, 22% of the Sjögren's syndrome sera, 6% of the Lyme's arthritis sera and 7% of the normal sera were rheumatoid factor positive. No correlation between the levels of anti-GPI IgG antibodies and rheumatoid factor was observed, R=0.19. The results further confirmed that the presence of rheumatoid factor in the serum was not the cause of positive signal in the GPI assay since some RA sera were strongly positive for GPI but negative for rheumatoid factor and vice versa. The sera were also tested against the irrelevant antigens, HIV-1 gp 120 and bovine serum albumin (BSA), to assess any contribution of polyreactive antibodies to reactivity with GPI. No significance difference between the percentage of sera with reactivity against BSA in the different patient groups was observed and none of the sera reacted with HIV-1 gp120 (data not shown). The lack of correlation between reactivity with GPI and BSA or gp120, indicated that polyreactive behavior could not explain serum reactivity with GPI in the majority of RA patients. The binding of the RA sera to GPI was further confirmed by staining of Western blots of purified GPI. As shown in FIG. 1B the serum of RA patients stained a 55 kDa band on the Western blots of the purified GPI preparation separated under non-reducing conditions. In contrast, no binding to the Western blots was observed with any of the normal sera or by the secondary antibody alone.

### Cloning of anti-GPI IgG Antibodies from a Patient with RA using Phase Display

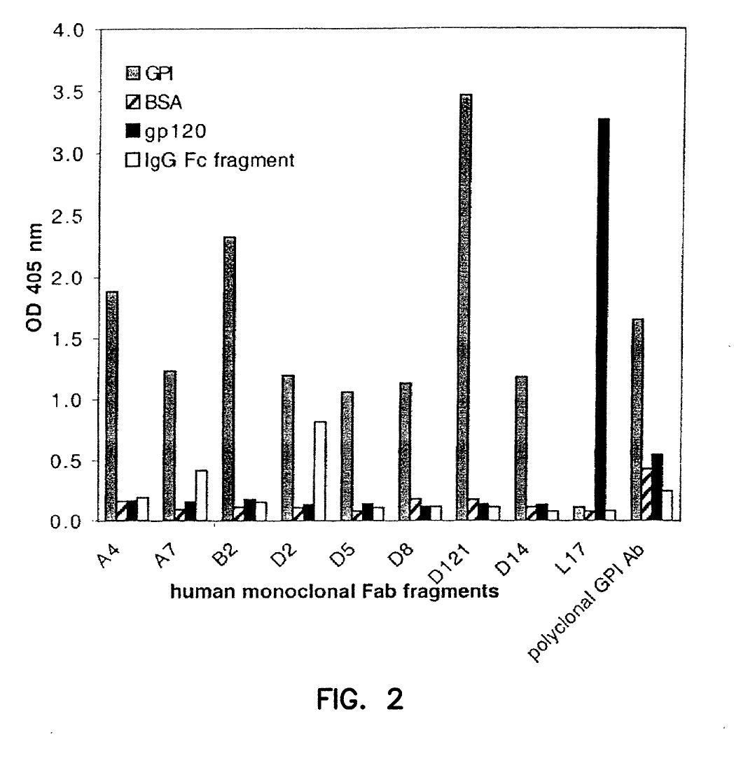

To further analyze the nature of the anti-GPI IgG antibodies, an IgG/kappa/lambda Fab phage display library of approximately 6×10⁶ members was constructed from the bone marrow RNA of one of the patients with RA who exhibited high serum titer to GPI. The antibody library was selected against purified GPI coated on to ELISA plates. Following 5 rounds of panning, a 1000-fold amplification in eluted phage was observed, indicating enrichment for antigen-binding clones. Three other antibody library, one generated from the bone marrow of a patient with systemic lupus erythematosus (SLE) with very low serum titer for GPI and two generated from the bone marrows of a healthy individuals with no indication of autoimmune disease or serum titer for GPI were also panned on GPI as controls. No enrichment in eluted phage was observed with the control libraries; rather, the phage titer decreased for each round of selection and did not amplify after the fourth round of selection. Subsequent ELISA screening of supernatants from 50 clones from the last round of selection with the RA library yielded 12 antibody Fab fragments that exhibited strong binding to GPI. Binding was specific in that no reactivity with HIV-1 gp120, BSA or the Fc part of IgG was found (FIG. 2). Sequencing of the DNA encoding the heavy chain variable region of these Fabs revealed that 7 clones were unique (FIG. 3A). The additional sequences were repeats of these sequences. A group of 3 Fabs A4, D2, and D121, consisting of somatic variants of one another and presumably evolved from a common ancestor, was identified (FIG. 3A). The anti-GPI IgG antibodies appear to result from an antigen-driven response The human monoclonal anti-GPI antibodies were examined to determine whether they exhibited signs of being evolved as a result of an antigen-driven response. The variable heavy and light chain genes of the IgG-derived anti-GPI Fab fragments were compared with the closest germline sequences in the GenBank database. Since previous studies have shown that the antibody heavy chain is the major contributor to antigen binding in many instances, detailed analysis was focused on this chain (FIG. 3AH). The light chain sequences are shown in FIG. 3AL. As shown in FIGS. 3B and 3C, all the variable heavy chain region genes of the anti-GPI IgG Fabs were significantly mutated, with nucleotide and amino acid homologies to the closest germline in the range of 79-95% (average 83%) and 67-91% (average 89%), respectively, characteristic of an affinity-matured antibody response. Interestingly, the two Fabs, A4 and B2, which exhibited the strongest binding to GPI, were the most somatically mutated. Further, the variable heavy chain genes exhibit high replacement (R) to silent (S) mutation ratio (R/S ratio) for the complementarity determining regions (CDRs) (CDR1 and 2) compared to the framework regions (FRs) (FR1, 2, and 3) (FIG. 3B).

Autoantibodies involved in an active immune response generally exhibit high affinity for their autoantigen. Therefore, we next determined the kinetic constants for the interaction of selected anti-GPI Fabs and GPI by surface plasmon resonance. The values measured for Fab B2 were kₒₙ=1.2×10⁴ M⁻¹s⁻¹, k_{off}=1.1×10⁻⁴s⁻¹, resulting in a dissociation constant (K_{d}) of 9.6×10⁻⁹ M. For Fab A4 the values were kₒₙ=4.3 10⁴ M⁻¹s⁻¹, k_{off}1.1×10⁻³ s⁻¹, resulting in a K_{d} of 2.4×10⁻⁸ M. Thus, the high degree of somatic mutation, the high R/S ratio, the intraclonal variants, and the affinity for the autoantigen indicate that these anti-GPI IgG antibodies are derived from bone marrow plasma/B cells involved in an active immune response.

### Synovial Fluid of RA Patients with Active Arthritis also Contains High Levels of Anti-GPI IgG Antibodies

To test whether anti-GPI IgG antibodies are present in the synovial fluid of RA patients as well as in serum, 24 patients with active RA were investigated, 29 patients with osteoarthritis and 2 normal individuals. Synovial fluids were titrated for binding to GPI by ELISA to assess the levels of antibodies present. Again, AP-conjugated F(ab')₂ fragment of a goat anti-human IgG Fc-specific antibody was used as secondary antibody to avoid problems of rheumatoid factor binding in the synovial fluid samples. FIG. 6 shows binding of IgG antibodies in synovial fluid diluted 1:200 to GPI. Synovial fluid from 8 RA patients exhibited high levels of anti-GPI IgG antibodies, whereas none of the 29 synovial fluid samples from patients with osteoarthritis or the 2 samples from normal joints exhibited IgG reactivity with GPI above the cut-off. Positivity was defined as an OD 405 value more than 2 standard deviations above the mean of the osteoarthritis values to GPI (>1.06) at a synovial fluid diluted 1:200. To evaluate specificity, the synovial fluid samples were also tested for IgG antibodies against the control antigens HIV-1 gp120 and BSA. None of the samples contained any IgG anti-gp120 or anti-BSA reactivity (data not shown). From two RA patients where both synovial fluid and serum were obtained, the anti-GPI titer in the two compartments was compared. Although the titer in the synovial fluid was slightly higher than in the serum, the difference was not significant.

The antigen GPI is found at high concentrations in both the serum and synovial fluid of RA patients

The concentration of GPI in the sera of RA patients was measured, Sjögren's syndrome patients and normal healthy individuals (FIG. 7). The serum GPI levels in healthy individuals has been reported to be 0.04-0.15 U/ml, within 95% confidence limits ¹³. In agreement with these results, sera of healthy individuals studied here had levels of 0.016-0.148 U/ml. In contrast, patients with RA exhibited significantly higher concentrations of GPI, ranging from 0.083-0.545 U/ml. All patients with Sjögren's syndrome except one exhibited normal serum levels of GPI. The mean level of GPI in the RA patient group (0.210+/−0.139 U/ml) was significantly higher than the normal healthy donor group (0.069+/−0.048 U/ml)(p<0.0001) or the Sjögren's syndrome group (0.094+/−0.049 U/ml)(p<0.0001). Interestingly, a significant positive correlation between levels of GPI and anti-GPI IgG antibodies in the RA sera was observed, R=0.79 (p=0.026). In addition, the GPI concentration in synovial fluid of the RA patients with active arthritis and patients with osteoarthritis was also measured (FIG. 7). The synovial fluid of the RA patients contained an even higher concentration of GPI than the RA sera (p=0.005). In contrast, the synovial fluid from the patients with osteoarthritis exhibited the same level of GPI as normal sera (p=0.1). The mean level of GPI in the RA patient group (0.431+/−0.049 U/ml) was significantly greater than the osteoarthritis group (0.060+/−0.052 U/ml)(p<0.0001).

### GPI-Containing Immune Complexes are Found in the Svnovial Fluid of Patients with Active RA Arthritis

To evaluate whether GPI and anti-GPI antibodies were present in the synovial fluid as immune complexes, fluid was fractionated by size exclusion chromatography using an S-200 column. The fractions were coated on ELISA wells and the binding of the anti-human GPI and anti-human IgG Fc antibodies, respectively, to the different fractions evaluated (FIG. 8A). The fractions were also further separated by SDS-PAGE and transferred to Immobilon P membranes. Subsequently, Western blots were stained with the anti-GPI antibody and with the anti-human IgG Fc antibody (FIG. 8B and C). As shown in FIG. 8A, ELISA analysis revealed binding of the anti-GPI antibody corresponding to two peaks. The first peak corresponded to the first three fractions after the void volume and exhibited a molecular mass of 200 kDa and higher (FIG. 8B). The second GPI peak corresponded to free GPI with a molecular mass of approximately 55 kDa. Similarly, the anti-human IgG Fc antibody bound to the first fractions after the void volume in the ELISA and stained complexes higher than 200 kDa. The staining of the following fractions corresponded predominantly to a band of 150 kDa, the molecular mass of free IgG (FIG. 8C). Non-fractionated synovial fluid was also centrifuged to evaluate whether the GPI-containing immune complexes could be precipitated. As shown in FIG. 8B, Western blots of high-speed centrifuged (C) or non-centrifuged (UC) synovial fluid stained with the anti-GPI antibody demonstrated that the high molecular weight broad band was significantly reduced following centrifugation, whereas the 55 kDa band, corresponding to free GPI, remained the same. In summary the data indicates that GPI and anti-GPI antibodies are found in the synovial fluid as immune complexes as well as in free forms.

### GPI is Present on the Endothelial Cell Surface of Synovial Arterioles and Capillaries and on the Surface of the Svnovial Lining

To determine whether GPI was distributed differently in synovial tissue from patients with active RA synovitis as compared to patients with osteoarthritis or healthy individuals, fresh frozen synovial tissue from these three groups was obtained and immunohistochemical analysis on cryostat tissue sections performed using a rabbit anti-GPI antibody (FIG. 9). The rabbit anti-GPI was used instead of the monoclonal human anti-GPI Fab fragments since the use of the secondary FITC labeled goat anti-human IgG F(ab')₂ antibody caused high background due its detection of endogenous immunoglobulin.

Laser scanning confocal microscopy of RA synovium using the anti-GPI antibody exhibited a faint diffuse cytoplasmic staining of all cells that was most pronounced in muscle cells, in accordance with the fact that GPI is ubiquitously expressed in all cells and higher levels are found in smooth muscle tissue. However, an additional intense staining of the surface of the endothelial cells of the synovial arterioles (FIG. 9A-C arrowhead) and some capillaries (FIG. 9C, arrow) was observed. In contrast, no staining of the surface of endothelial cells of venules in RA synovial tissue was found (FIG. 9 A, open arrow). No damage to the stained synovial arterioles or capillaries was observed by morphologic examination of adjacent tissue sections stained with hematoxylin-eosin. Intense patchy staining corresponding to the surface lining of the hypertrophic synovium was also observed (FIG. 9F-H) and particularly strongly at the hypertrophic synovial villous formations (FIG. 9G, open arrow). The staining was located intracellularly in the upper layer of some synoviocytes (FIG. 9G-H) and extracellularly. The extracellularly patchy staining appeared to be material precipitated (perhaps immune complexes) on the surface lining (FIG. 9F, arrow). No staining of the RA synovial tissue was observed with the secondary antibody alone (FIG. 9D and I). Examination of the synovium from osteoarthritis patients (FIG. 9E and J) and a healthy individual revealed faint background staining in most cells, but no intense staining either at the endothelial cell surface or on the synovial lining. In addition, GPI staining of endothelial cells of small blood vessels were examined in different tissues obtained from patients with RA, including pericardium, skin, connective tissue and endometrium. While staining was observed on the surface of the endothelial cells of the small synovial blood vessels, none was observed in the blood vessels of pericardium, skin, connective tissue and endometrium obtained from the same RA patients. Interestingly, high level GPI staining was found in keratinocytes of the skin.

### Library Construction and Phage Selection

Preparation of RNA from bone marrow cells and subsequent construction of IgGI μ/λ Fab libraries using the pComb3 M13 surface display system has been described ^{35;36} Human materials including bone marrow were obtained according to human subject protocol no. 98-254 approved by The Scripps Research Institute's Human Subjects Committee. For antibody selection, 4 phage libraries generated from a donor with RA, a donor with SLE and two healthy donors were panned on GPI (Sigma, St. Louis, Mo.) purified from rabbit muscle ³⁷ and coated at 5 μg/ml on ELISA plates overnight. The 558 amino acid protein sequence of rabbit GPI shows 93% amino acid sequence identity to human GPI. The variable residues are concentrated at the N-terminus (eight variable positions between residues 15 and 35) and in the C-terminus (last five residues). In brief, phage resuspended in phosphate buffered saline (PBS) containing 1% bovine serum albumin (BSA) were incubated for 2 hr at 24° C. Unbound phage were removed by washing 10 times with PBS containing 1% BSA. Bound phage, enriched for those bearing antigen-binding surface Fabs, were eluted with 0.2 M glycine-HCl buffer pH 2.2. The eluted phages were amplified by infection of E. coli and superinfection with M13 helper phage. The panning procedure was repeated 5 times, after which phagemid DNA was prepared from the last round and the gene III fragment was removed by treatment with the enzymes NheI and SpeI, followed by re-ligation. The reconstructed phagemid was used to transform XL1-Blue cells to produce clones secreting soluble Fab fragments.

### Purification of Fabs and ELISA Analysis

Fabs were purified from bacterial supernatants by affinity chromatography, as previously described, with minor modifications ^{38;39}. In brief, E. coli containing the appropriate clone were inoculated into liter cultures of superbroth containing carbenicillin (50 μg/ml), tetracycline (10 μg/ml) and MgCl₂ (20 mM), and grown at 37° C., with shaking, for 6 h. Protein expression was then induced with 2 mM isopropyl -D-thiogalactopyranoside and cells grown at 30° C. overnight. Soluble Fab was purified from bacterial supernatants by affinity chromatography using a rabbit anti-human Fab antibody coupled to protein A Sepharose Fast Flow matrix (Amersham Pharmacia Biotech Inc., Piscataway, N.J.). The column was washed with PBS, antibody eluted in 0.2 M glycine-HCl buffer pH 2.2 and immediately brought to neutral pH with 1 M Tris-HCl, pH 9.0. To assess specificity, supernatants were screened by ELISA against GPI and a panel of unrelated antigens, including HIV-1 gp 120 (IIIB strain) (Intracell, Issaquah, WA), Fc fragment of Ig and BSA. Human Fabs or polyclonal rabbit anti-GPI antibody were incubated with the test antigen for 2 hr at 37° C., followed by washing 10 times with PBS-0.05% Tween. The specificity of the rabbit anti-GPI antibody has been reported by Raz and colleagues 9. Detection of bound human Fabs and rabbit antibody was carried out with alkaline phosphatase (AP)-labeled goat anti-human IgG F(ab')₂ antibody (Pierce, Rockford, Ill.) or AP-labeled goat anti-rabbit IgG antibody (Pierce) diluted 1:500 in PBS, and visualized with nitrophenol substrate (NPP substrate) (Sigma) by reading absorbance at 405 nm.

Sera from 69 patients diagnosed with RA, 17 patients with Lyme's arthritis, 22 patients with primary Sjögren's syndrome, and 107 healthy normal volunteers were tested for binding to purified GPI. In addition, synovial fluid from 24 patients with active RA, 29 patients with joint degenerative disease (osteoarthritis) and 2 normal individuals (obtained 12-hrs post-mortem) were also tested for binding to purified GPI. Diagnoses of RA, Lyme's arthritis and Sjögren's syndrome were defined according to the classification criteria of the American College of Rheumatology. The RA patients and healthy normal volunteers were age and gender matched. No correlation between GPI positivity and age or gender was observed; as an example the normal donor group included 5 females ≧55-years of age whose sera all were negative for GPI. The sera, dilutions from 1:12600-1:25, were incubated with the test antigen for 2 hr at 37° C., washed with PBS-0.05% Tween 20 and detected with AP-labeled F(ab')₂ fragment of goat anti-human IgG Fc-specific antibody (Jackson, West Grove, Pa., 1:1000 in PBS). After 5 washes with PBS-0.05% Tween 20, bound antibody was visualized with NPP substrate and read at 405 nm. To assure standardized conditions for the anti-GPI ELISAs, titrations of two standard control sera are always included; one having high anti-GPI titer and one having moderate anti-GPI titer. Testing for significant differences between means was performed with the unpaired Student's t test whereas testing for significant correlation between GPI and anti-GPI antibody concentration was performed with the Spearman rank test. The sera and synovial fluids were also screened for binding to rheumatoid factor, HIV-1 gp120 (IIIB strain), and BSA by ELISA as described above.

### Nucleic Acid Sequencing

Nucleic acid sequencing was carried out on a 373A automated DNA sequencer (ABI, Foster City, Calif.) using a Taq fluorescent dideoxy terminator cycle sequencing kit (ABI). Comparison to reported Ig germline sequences from Genbank and EMBL was performed using the Genetic Computer Group (GCG) Sequence Analysis program.

### Enzymatic Assay for GPI Activity

The GPI concentration of the samples was measured using a spectrophotometric assay measuring the GPI enzymatic activity 13, according to manufacturer's guidelines (Sigma). In brief, 250 mM glycylglycine buffer, pH 7.4 at 25° C., 100 mM D-fructose 6-phosphate solution, 20 mM -nicotinamide adenine dinucleotide phosphate solution, 100 mM magnesium chloride solution, and glucose-6-phosphate dehydrogenase enzyme solution (50 units/ml) were mixed in a ratio of 20:5:1:1:1 and allowed to equilibrate to 25° C. Absorbance at 340 nm was monitored until it reached a constant level using a thermostatted spectrophotometer, when a 33 μl sample (sera (1:50), synovial fluid (1:50) or purified rabbit GPI) was added, making the total reaction volume 1 ml. After mixing the sample, the increase in A340 nm was recorded every 30 sec for 10 min. The A 340 nm/min was measured using the maximum linear rate and the Units/ml calculated. As control, concentrations of purified GPI were tested, confirming over 80% assay accuracy.

### Gel Permeation Chromatography

Synovial fluid, diluted 1:10 in PBS, was separated by gel permeation chromatography using a size separation column (Sephadex S-200, Amersham Pharmacia) on an AKTA Explorer instrument (Amersham Pharmacia). The synovial fluid was separated at a flow rate of 0.1 μl/min and 1 ml fractions collected.

### SDS-PAGE and Western Blotting

Synovial fluid or purified GPI was mixed with 2x sample buffer (4% SDS, 0.2% bromophenol blue, 20% glycerol in 100 mM Tris buffered saline) and boiled for 5 min. The samples were electrophoresed under reducing or non-reducing conditions on a 7.5% solving gel (Bio-Rad) and the proteins electroblotted onto Immobilon P (Millipore). The Immobilon sheet was blocked in 5% nonfat dry milk in PBS for 1 hr, and incubated with the antibody overnight at 4° C. After repeated washing (5×5 min), bound antibody was detected with horse-radish peroxidase (HRP)-labeled goat anti-human Fab antibody (Pierce), HRP-labeled F(ab')₂ fragment of goat anti-human IgG Fc-specific antibody (Jackson) or HRP-labeled goat anti-rabbit IgG antibody (Pierce) incubated for 1 hr and visualized by chemiluminescent substrate (Supersignal Substrate, Pierce) and autoradiographic film (Eastman Kodak company, Rochester, N.Y.). Control staining of Western blots omitting the primary antibody was included.

### Immunohistochemical Analysis using Confocal Laser Scanning Microscopy

Fresh frozen and formalin-fixed, paraffin embedded, tissue was obtained from joints of patients with active RA, joint degenerative disease (non-RA arthritis) and otherwise healthy individuals who had undergone arthroscopic surgery for traumatic injury-related disease. In addition, tissues including pericardium, skin, connective tissue and endometrium from RA patients who had undergone surgery for additional diseases were also examined. Blocks were cut into 5 μm sections, which were dried overnight. The formalin-fixed, paraffin-embedded, sections were deparaffinized in xylene and rehydrated through graded ethanols. Frozen tissue sections were fixed by ice-cold 96% ethanol at 4° C. for 5 min. After a brief rinse in PBS, sections were blocked with 5% normal goat serum followed by incubation with rabbit anti-GPI antibodies (diluted 1:50 in PBS). The slides were washed 3 times for 5 min. with PBS and incubated with FITC-labeled goat antirabbit IgG antibody (1:500 in PBS, Sigma) and propidium iodide (Sigma) for 1 hr at room temperature. The slides were again washed with PBS for 15 min at room temperature and anti-fading reagent Slow Fade in PBS/glycerol (Molecular Probes, Eugene, Oreg.) was added. Staining of cells was evaluated by immunofluorescence microscopy or confocal laser scanning microscopy. As controls, all experiments were carried out omitting the primary antibody or included rabbit serum instead of the primary antibody. In addition, parallel sections were stained with hematoxylin-eosin for morphologic analysis.

### Surface Plasmon Resonance to Measure Fab Binding Affinities

The kinetics of Fabs binding to GPI was determined by surface plasmon resonance-based measurements using the BlAcore instrument (Pharmacia). Purified GPI (15 μl at a concentration of 10 μg/ml in acetate buffer, pH 4.0) was coupled to a CM5 sensor chip using N-hydroxysuccinimide (NHS)/N-ethyl-N'-(3-dimethylaminopropyl) carbodiimide amine coupling chemistry. Typically, 3000 resonance units were immobilized. The association and dissociation rate constants, kon and koff, were determined under continuous flow rate of 10 μl/min using a range of concentrations (

31-500 nM) of Fabs, as described ⁴⁰. Association was determined from a plot of (ln(do/DT)/t) versus concentration. Equilibrium association and dissociation constants were deduced from the rate constants.

### Citations For Examples and Investigation Sections

5. Korganow, A-S et al. From systemic T cell self-reactivity to organ-specific autoimmune disease via immunoglobulins, Immunity, 10, 451-461 (1999).

6. Kouskoff, V. et al. Organ-specific disease provoked by systemic autoimmunity. Cell 87, 811-822 (1996).

7. Chaput, M. et al. The neurotrophic factor neuroleukin is 90% homologous with phosphohexose isomerase. Nature 332, 454-455 (1988).

8. Faik, P., Walker, J. I. H., Redmill, A. A. M. & Morgan, M. J. Mouse glucose-6-phosphate isomerase and neuroleukin have identical 3′ sequences. Nature 332, 455-457 (1988).

9. Watanabe, H., Takehana, K., Date, M., Shinozaki, T. & Raz, A. Tumor cell autocrine motility factor is the neuroleukin/phosphohexose isomerase polypeptide. Cancer Res. 56, 2960-2963 (1996).

10. Xu, W., Seiter, K., Feldman, E., Ahmed, T. & Chiao, J. W. The differentiation and maturation mediator for human myeloid leukemia cells shares homology with neuroleukin or phosphoglucose isomerase. Blood 87, 4502-4506 (1996).

11. Gurney, M., Heinrich, S. P., Lee, M. R. & Yin, H. Molecular cloning and expression of neuroleukin, a neurotrophic factor for spinal and sensory neurons. Science 234, 566-574 (1986).

12. Gurney, M. E. et al. Neuroleukin: a lymphokine product of lectin-stimulated T cells. Science 234, 574-581 (1986).

13. Rowan, R. M. The assay of phosphoglucose isomerase in human serum. Med. Lab. Sciences 35, 155-166 (1978).

14. Matsumoto, I., Staub, A., Benoist, C. & Mathis, D. Arthritis provoked by linked T and B cell recognition of a glycolytic enzyme. Science 286, 1732-1735 (1999).

15. Benner, R., Hijmans, W., and Haajiman, J. J. The bone marrow: the major source of serum immunoglobulins, but still a neglected site of antibody formation. Clin. Exp. Immunol. 46, 1-7 (1981).

16. Koch, A. E. Angiogenesis: implications for rheumatoid arthritis. Arthritis Rheum. 41, 951-962 (1998).

17. Jalkanen, S., Steere, A. C., Fox, R. I. & Butcher, E. C. A distinct endothelial cell recognition system that controls lymphocyte traffic into inflamed synovium. Science 233, 556-558 (1986).

18. Ferrara, N., Houck, L., Jakeman, L. & Leung, D. W. Molecular and biological properties of the vascular endothelial cell growth factor family of proteins. Endocrinol. Rev. 13, 18-32 (1992).

19. Jackson , J. R., Minton, J. A., Ho, M. L., Wei, N. & Winkler, J. D. Expression of vascular endothelial growth factor in synovial fibroblasts is induced by hypoxia and interleukin lbeta. J. Rheumatol. 24, 1253-1259 (1997).

20. Watanabe, H. et al. Purification of human tumor cell autocrine motility factor and molecular cloning of its receptor. J. Biol. Chem. 266, 13442-13448 (1991).

21. Silletti, S. & Raz, A. Regulation of autocrine motility factor receptor expression in tumor cell locomotion and metastasis. Current Topics Microbiol. Immunol. 213, 137-169 (1996).

22. Oldstone, M. B. A. Molecular mimicry and autoimmune disease. Cell 50, 819-820 (1987).

23. Wucherpfenning, K. W. & Strominger, J. L. Molecular mimicry in T cell-mediated autoimmunity: viral peptides activate human T cell clones specific for myelin basic protein. Cell 80, 695-705 (1995).

24. Rocken, M., Urban, J. F., & Shevach, E. M. Infection breaks T-cell tolerance. Nature 359, 79-82 (1992).

25. Ohashi, P. S. et al. Ablation of “tolerance” and induction of diabetes by virus infection in viral antigen transgenic mice. Cell 65, 305-317 (1991).

26. Caton, A. J., Swartzentruber, J. R., Kuhl, A. L., Carding, S. R., & Stark, S. E. Activation and negative selection of functionally distinct subsets of antibodysecreting cells by influenza hemagglutinin as a viral and a neo-self antigen. J. Exp. Med. 183, 13-26 (1996).

27. Goodnow, C. C., Brink, R., & Adams, E. Breakdown of self-tolerance in anergic B lymphocytes. Nature 352, 532-536 (1991).

28. Fulcher, D. A. et al. The fate of self-reactive B cells depends primarily on the degree of antigen receptor engagement and availability of T cell help. J. Exp. Med. 183, 2313-2328 (1996).

29. Cooke, M. P. et al. Immunoglobulin signal transduction guides the specificity of B cell-T cell interactions and is blocked in tolerant self-reactive B cells. J. Exp. Med. 179, 425-438 (1994).

30. Baumann, M., Jezussek, D., Richter, R. T. & Brand, K. Variants of phosphohexose isomerase in gastrointestinal and mammary carcinoma: isoelectric focusing patterns of normal and tumor tissues derived from surgical specimens of the same patient. Cancer Res. 48, 2998-3001 (1988).

31. Gomm, S. A., Keevil, B. G., Thatcher, N., Hasleton, P. S. & Swindell, R. S. The value of tumour markers in lung cancer. Br. J. Cancer 58, 797-804 (1988).

32. Filella, X., Molina, R., Jo, J., Mas, E. & Ballesta, A. M. Serum phosphohexose isomerase activities in patients with colorectal cancer. Tumor. Biol. 12, 360-367 (1991).

33. Burton, D. R. & Woof, J. M. Human antibody effector function. Adv. Immunol. 51, 118-131 (1992).

34. Maini, R. et al. Infliximab (chimeric anti-tumor necrosis factor alpha monoclonal antibody) versus placebo in rheumatoid arthritis patients receiving concomitant methotrexate: a randomised phase III trial. ATTRACT Study Group. Lancet 354, 1932-1939 (1999).

35. Barbas, C. F. I., Kang, A. S., Lemer, R. A. & Benkovic, S. J. Assembly of combinatorial antibody libraries on phage surfaces: The gene III site. Proc. Natl. Acad. Sci. USA 88, 7978-7982 (1991).

36. Burton, D. R. et al. A large array of human monoclonal antibodies to type 1 human immunodeficiency virus from combinatorial libraries of asymptomatic seropositive individuals. Proc. Natl. Acad. Sci. USA 88, 10134-10137 (1991).

37. Phillips, T. L., Talent, J. M. & Gracy, R. W. Isolation of rabbit muscle glucosephosphate isomerase by a single-step substrate elution. Biochim. Biophys. Acta 429,624-628 (1976).

38. Barbas, C. F. I. et al Human monoclonal Fab fragments derived from a combinatorial library bind to respiratory syncytial virus F glycoprotein and neutralize infectivity. Proc. Natl. Acad. Sci. USA 89, 10164-10168 (1992).

39. Ditzel, H. J. et al. Neutralizing recombinant human antibodies to a confornational V2- and CD4-binding site-sensitive epitope of HIV-1 gp 120 isolated by using an epitope-masking procedure. J. Immunol. 154, 893-906 (1995).

40. Karlsson, R. A., Michaelsson, A. & Mattsson, L. Kinetic analysis of monoclonal antibody-antigen interactions with a new biosensor based analytical system. J. Immunol. Methods 145, 229-240 (1991).

All publications, patents and patent applications are incorporated herein by reference. While in the foregoing specification, this invention has been described in relation to certain preferred embodiments thereof, and may details have been set forth for the purposed of illustration, it will be apparent to those skilled in the art that the invention includes additional embodiments and that certain of the details described therein may be varied considerable without departing from the basic principles of the invention.

What is claimed is:

1. An immunopolypeptide that binds to human glucose-6-phosphate isomerase with a dissociation constant of no more than about 10⁻⁷.

2. An isolated immunoglobulin antibody that specifically binds to human glucose-6-phosphate isomerase

3. An immunopolypeptide comprising at least one CDR sequence selected from the group consisting of SEQ ID NO's: 15-56 or a significant homolog thereof.

4. An immunopolypeptide according to claim 3 having a triplet of CDR sequences.

5. An immunopolypeptide according to claim 4 wherein each CDR of the triplet is separated from other CDR's by a spacer amino acid sequence.

6. An immunopolypeptide according to claim 5 wherein the spacer amino acid sequence is a framework region sequence having an amino acid sequence selected from the group consisting of SEQ ID NO's: 57-108 or a significant homolog thereof.

7. An immunopolypeptide according to claim 6 wherein the CDR's of the triplet are selected from either a light chain group or a heavy chain group.

8. An immunopolypeptide according to claim 7 wherein the CDR's are matched according to their Fab source.

9. An immunopolypeptide according to claim 8 wherein the framework region sequence is matched to the Fab source of the CDR triplet.

10. An immunopolypeptide according to claim 9 wherein the amino acid sequence is a V_{L} or V_{H} fragment of the Fab source of the matched CDR triplet and framework regions.

11. An immunopolypeptide having an amino acid sequence substantially homologous to a sequence selected from the group consisting of SEQ ID NO's: 1-14.

12. An immunopolypeptide according to claim 11 which is a combination of V_{L} and V_{H}.

13. An immunopolypeptide according to claim 11 which further includes at least one constant consensus region.

14. An immunopolypeptide according to claim 13 which is a combination of a light and heavy chain fragment.

15. An immunopolypeptide according to claim 14 which is an Fab, Fab', F(ab')₂, Fd, scFv or Fv fragment.

16. An anti-GPI monoclonal antibody having CDR and framework segments with significant homology to the amino acid sequences set forth in SEQ ID NO's: 15-108.

17. A immunopolypeptide encoded in a bacteriophage that is deposited with the ATCC.

18. An immunopolypeptide Fab fragment having a light variable chain amino acid sequence selected from the group consisting of SEQ ID NO's: 1-7 and a heavy variable amino acid sequence selected from the group consisting of SEQ ID NO's: 8-14.

19. An immunopolypeptide Fab fragment having its CDR amino acid sequences of its light chain selected from the group consisting of SEQ ID NO's: 36-56 and its CDR amino acid sequence of its heavy chain sequence selected from the group consisting of SEQ ID NO's: 15-35.

20. An anti-idiotypic antibody that specifically binds with anti-glucose-6-phosphate isomerase antibody.

21. An anti-idiotypic antibody according to claim 20 which binds with a hypervariable region segment of anti-glucose-6-phosphate antibody

22. A second immunopolypeptide that specifically binds with anti-6-phosphate isomerase antibody.

23. A second immunopolypeptide according to claim 22 that specifically binds with a variable region segment of anti-6-phosphate isomerase.

24. An antisense oligonucleotide that specifically hybridizes with a polynucleotide encoding anti-glucose-6-phosphate isomerase antibody or encoding glucose-6-phosphate isomerase.

25. An antisense oligonucleotide according to claim 24 having a non-natural modification.

26. An antisense oligonucleotide according to claim 25 having at least one thiophosphate group, a base alkylation group or a non-natural base group.

27. A conjugate of human glucose-6-phosphate isomerase covalently bonded to, complexed with, or associated with, a cytotoxic agent.

28. A composition comprising immobilized human glucose-6-phosphate isomerase.

29. A nucleotide sequence encoding an immunopolypeptide according to claim 1.

30. A nucleotide sequence having a sequence selected from the group consisting of SEQ ID NOs: 109-122.

31. A nucleotide sequence encoding an anti-glucose-6-phosphate isomerase antibody.

32. A nucleotide sequence encoding a humanized chimeric monoclonal antibody according to claim 20.

33. A pharmaceutical composition comprising an immunopolypeptide of claim 1 and a pharmaceutically acceptable carrier.

34. A pharmaceutical composition comprising an anti-idiotypic antibody according to claim 20 and a pharmaceutically acceptable carrier.

35. A pharmaceutical composition comprising a second immunopolypeptide according to claim 22 and a pharmaceutically acceptable carrier.

36. A pharmaceutical composition comprising an antisense oligonucleotide according to claim 24 and a pharmaceutically acceptable carrier.

37. A pharmaceutical composition comprising a conjugate according to claim 27 and a pharmaceutically acceptable carrier.

38. A method for diagnosis of autoimmune disease comprising determining the presence of an immune complex formed by combining the blood sera of a patient with human glucose-6-phosphate isomerase.

39. A method for treatment of a patient having autoimmune disease comprising administering to the patient an effective amount of an immunopolypeptide according to claim 1.

40. A method for treatment of a patient having autoimmune disease comprising administering to the patient an effective amount of a humanized chimeric monoclonal antibody according to claim 20.

41. A method for treatment of a patient having autoimmune disease comprising administering to the patient an effective amount of a second immunopolypeptide according to claim 22.

42. A method for treatment of a patient having autoimmune disease comprising administering to the patient an effective amount of a conjugate according to claim 27.

43. A method for treatment of a patient having autoimmune disease comprising administering to the patient an effective amount of an antisense oligonucleotide according to claim 24.

44. A method for treatment of a patient having autoimmune disease comprising filtering the patient's blood extracorporeally through a filter system containing immobilized human glucose-6-phosphate isomerase.

45. A method for treatment of a patient having autoimmune disease comprising administering to the patient an effective desensitizing a mount of human glucose-6-phosphate isomerase.

46. An antisense oligonucleotide according to claim 24 which hybridizes with the nucleotide sequence encoding the antibody.
